(19) United States
(12) Patent Application Publication
(10) Pub. No.: US 2024/0352483 A1
(43) Pub. Date: Oct. 24, 2024

(54) MODIFIED REP-CAP PLASMIDS AND USES THEREOF

(71) Applicant: Decibel Therapeutics, Inc., Tarrytown, NY (US)

(72) Inventors: Sandra Danielle Abbott, Danvers, MA (US); Noah Druckenbrod, Dedham, MA (US); Tyler Gibson, Boston, MA (US); Kevin Lebo, Weymouth, MA (US)

(21) Appl. No.: 18/626,156

(22) Filed: Apr. 3, 2024

Related U.S. Application Data
(60) Provisional application No. 63/545,351, filed on Oct. 23, 2023.
(60) Provisional application No. 63/456,632, filed on Apr. 3, 2023.

(51) Int. Cl. C12N 15/86; C07K 14/47

(52) U.S. Cl. CPC C12N 15/86; C07K 14/47; C12N 2750/14143; C12N 2750/14152

(57) ABSTRACT

The disclosure provides modified rep-cap plasmids containing a tetracycline-inducible expression system that can be used to produce recombinant AAV1 vectors.

This application claims priority to U.S. Application Ser. No. 63/545,351, filed Oct. 23, 2023 and U.S. Application Ser. No. 63/456,632, filed Apr. 3, 2023. These applications are hereby incorporated by reference.

### FIELD OF THE INVENTIONS

The present inventions relate to modified Rep-Cap plasmids and uses of modified Rep-Cap plasmids, such as to produce recombinant adeno-associated virus (rAAV). The rAAV can comprise a transgene of interest, which includes any gene that is desired to be expressed and can fit within an AAV capsid.

### BACKGROUND

Research and clinical applications of recombinant adeno-associated virus have significantly increased in recent years alongside regulatory approvals of rAAV gene therapy products. For gene therapy to be effective, the rAAV vector must be present at a sufficient concentration to exert a therapeutic effect. The standard approach used to produce rAAV vectors leads to a concentration that is too low for patient treatment. Therefore, to reach an effective dose in a reasonable volume, the rAAV vectors must be further concentrated. Concentration of the vectors may also lead to concentration of contaminants or impurities, and, thus, is not without drawbacks. Accordingly, there is a need for new approaches to increase AAV vector production.

### REFERENCE TO ELECTRONIC SEQUENCE LISTING

The application contains a Sequence Listing, which has been submitted electronically in .XML format and is hereby incorporated by reference. The .XML copy, created on Mar. 27, 2024, is named “11635.xml” and is 68,473 bytes in size. The sequence listing contained in this .XML file is part of the specification and is hereby incorporated by reference.

### SUMMARY OF THE INVENTIONS

The present inventions provide modified adeno-associated virus (AAV) rep-cap plasmids and methods of use thereof to produce AAV vectors having an AAV capsid (for example, an AAV1 capsid). The modified rep-cap plasmids can contain a tetracycline-inducible expression system (for example, a Tet-Off or a Tet-On system) positioned between a polynucleotide encoding an AAV rep protein and a polynucleotide encoding an AAV capsid protein (for example, an AAV1 capsid protein). For example, the rep-cap plasmids can have an arrangement according to FIG. 1, 3, 5, 6 or 7, and can comprise on or more sequences set forth herein.

When a modified rep-cap plasmid is introduced into a cell along with a helper plasmid and a transgene plasmid, rAAV (for example, rAAV1) production is increased compared to rAAV (for example, rAAV1) production using a traditional rep-cap plasmid (for example, a rep-cap plasmid that lacks a tetracycline inducible expression system). The tetracycline-inducible expression system can be “turned off” or “inactivated” (for example, by exposing a plasmid containing a Tet-Off system to an inducer, such as doxycycline, or by maintaining a plasmid containing a Tet-On system in the absence of an inducer) to increase the percentage of full rAAV vectors produced (that is, rAAV vectors containing a transgene of interest to be expressed). Accordingly, the compositions and methods described herein can be used to produce rAAV vectors, such as rAAV1 vectors, for use in gene therapy (for example, for a gene therapy approach that would utilize AAV1 vectors, such as a gene therapy approach directed towards the central nervous system, heart, skeletal muscle, retinal pigment epithelium, or inner ear).

The inventions provide adeno-associated virus (AAV) rep-cap plasmid including, in a 5′ to 3′ orientation: (a) a polynucleotide encoding an AAV rep protein; (b) a tetracycline-inducible expression system; (c) an AAV promoter, such as p41 or TRE-tight; and (d) a polynucleotide encoding an AAV1 capsid protein.

The AAV rep protein can be an AAV2 rep protein.

The polynucleotide encoding an AAV rep protein can encode Rep78 and Rep52. Rep78 can have the sequence of SEQ ID NO: 1. Rep52 can have the sequence of SEQ ID NO: 2.

The polynucleotide encoding the AAV rep protein can have the sequence of SEQ ID NO: 3.

The tetracycline-inducible expression system can be a tetracycline-off (Tef-Off) system. The Tet-Off system can include, in a 5′ to 3′ orientation: (a) a first polynucleotide encoding a tetracycline trans-activator (tTA), wherein the tTA can include a tetracycline-repressor-derived DNA binding domain and a transcriptional activation domain; and (b) a second polynucleotide containing 1 to 12 tet operator (tetO) sequences. The first polynucleotide can encode a polypeptide having an amino acid sequence of SEQ ID NO: 5. The first polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to SEQ ID NO: 6 (for example, can have at least 80% sequence identity to SEQ ID NO: 6 and can encode a polypeptide having an amino acid sequence of SEQ ID NO: 5). The first polynucleotide can have the sequence of SEQ ID NO: 6. Each tetO sequence can have the sequence of SEQ ID NO: 4. Each tetO sequence can be separated from an adjacent tetO sequence by 0-20 nucleotides (for example, by 0, 1, 2, 3, 4, 5, 6, 7, 8, 9, 10, 11, 12, 13, 14, 15, 16, 17, 18, 19, or 20 nucleotides). The second polynucleotide can comprise eight tetO sequences. The second polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to SEQ ID NO: 7. The second polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 7 apart from the eight tetO sequences, each of which can have the sequence of SEQ ID NO: 4. The second polynucleotide can have the sequence of SEQ ID NO: 7. The Tet-Off system further can comprise a polyA signal sequence located between the first polynucleotide and the second polynucleotide.

The tetracycline-inducible expression system can be a Tet-On system. The Tet-On system can include, in a 5′ to 3′ orientation: (a) a first polynucleotide encoding a reverse tetracycline trans-activator (rtTA), wherein the rtTA can include a tetracycline-repressor-derived DNA binding domain (for example, a reverse tetracycline repressor) and a transcriptional activation domain; and (b) a second polynucleotide containing 1 to 12 tet operator (tetO) sequences. The first polynucleotide can encode a polypeptide having an amino acid sequence of SEQ ID NO: 20. The first polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to SEQ ID NO: 21 (for example, can have at least 80% sequence identity to SEQ ID NO: 21 and can encode a polypeptide having an amino acid sequence of SEQ ID NO: 20). The first polynucleotide can have the sequence of SEQ ID NO: 21. Each tetO sequence can have the sequence of SEQ ID NO: 4. Each tetO sequence can be separated from an adjacent tetO sequence by 0-20 nucleotides (for example, by 0, 1, 2, 3, 4, 5, 6, 7, 8, 9, 10, 11, 12, 13, 14, 15, 16, 17, 18, 19, or 20 nucleotides). The second polynucleotide can comprise eight tetO sequences. The second polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to SEQ ID NO: 7. The second polynucleotide can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 7 apart from the eight tetO sequences, each of which can have the sequence of SEQ ID NO: 4. The second polynucleotide can have the sequence of SEQ ID NO: 7. The Tet-On system further can comprise a polyA signal sequence located between the first polynucleotide and the second polynucleotide.

The p41 promoter can have the sequence of SEQ ID NO: 8.

The polynucleotide encoding an AAV1 capsid protein can encode a polypeptide having an amino acid sequence of SEQ ID NO: 9. The polynucleotide encoding an AAV capsid protein can have the sequence of SEQ ID NO: 10. The polynucleotide encoding an AAV capsid protein can have the sequence of SEQ ID NO: 11.

The rep-cap plasmid can include a polynucleotide sequence of SEQ ID NO: 12. The rep-cap plasmid can include a polynucleotide sequence of SEQ ID NO: 13. The rep-cap plasmid can include a polynucleotide sequence of SEQ ID NO: 22.

The present inventions also provide methods of manufacturing recombinant adeno-associated virus by introducing into a mammalian cell: (a) a rep-cap plasmid of any of the foregoing inventions, such as use of the p41 or TRE-tight promoters; (b) a helper plasmid containing one or more helper genes selected from E4, E2a and VA; and (c) a transgene plasmid including a transgene of interest flanked by inverse terminal repeats. The introducing step can be under conditions that permit formation of the recombinant adeno-associated virus. The methods further can include the step of collecting the recombinant adeno-associated virus. The transgene plasmid further can include a promoter operably linked to the transgene of interest. The mammalian cell can be a HEK-293 cell. The rep-cap plasmid can include a polynucleotide sequence of SEQ ID NO: 12. The rep-cap plasmid can include a polynucleotide sequence of SEQ ID NO: 13. The rep-cap plasmid can include a polynucleotide sequence of SEQ ID NO: 22. The transgene of interest can encode a protein that is endogenously expressed in the human inner ear. The transgene of interest can encode, for example, solute carrier family 26, member 4 (Pendrin), Otoferlin (OTOF), Stereocilin (STRC), Atonal BHLH Transcription Factor 1 (ATOH1), gap junction protein beta 2 (GJB2), or SRY-Box 2 (Sox2), for example. Other transgenes that can be expressed include, for example, GJB6; WFS1; COCH; EYA4; MYO7A; POU4F3; ACTG1; MY06; REST; NLRP3; COL11A1; TJP2; TBC1D24; SLC26A4; ELMOD3; EPSN; WHRN; IGF1; IGF1R; MYO15A; TMIE; TMC1; TMC2; TMPRSS3; OTOF; CDH23; GIPC3; USH1C; OTOG; TECTA; OTOA; PDCH15; CLDN14; WHRN; ESRRB; MYO3A; HGF, ILDR1, ADCY1; CIB2; MARVELD2; SOX2; SLC26A5; COL4A3; COL4A4; COL4A5; CLPP; PJVK; LRTOMT/COMT2; LOXHD1; TPRM; SYNE4; KCNJ10; PTPRQ; OTOGL; LHFPL5; A1PR2; CABP2; MET; GRXCR2; EPS8; CLIC5; EPS8L2; WBP2; ROR1; CLDN9; USH1G; PKHD1L1; DIAPH3; NDP; USH2A; CLRN1; SANS; HARS1; TRIOBP and other genes. A transgene of interest includes any gene that is desired to be expressed and can fit within an AAV capsid. More than one transgene of interest is possible if the totality of genes are of a combined size that can fit within an AAV capsid.

The viral yield can be at least 1×10¹¹ vg/ml (for example, 1×10¹¹ vg/mL, 2×10¹¹ vg/mL, 3×10¹¹ vg/mL, 4×10¹¹ vg/mL, 5×10¹¹ vg/mL, or greater, such as such as 1×10¹¹ vg/mL to 5×10¹¹ vg/mL, 1×10¹¹ vg/mL to 1×10¹² vg/mL, 1×10¹¹ vg/mL to 5×10¹² vg/mL, or 1×10¹¹ vg/mL to 1×10¹³ vg/mL) as measured using ddPCR of clarified lysate.

The inventions also provide methods of manufacturing recombinant adeno-associated virus by introducing into a mammalian cell: (a) a rep-cap plasmid including, in a 5′ to 3′ orientation: (i) a polynucleotide encoding an AAV rep protein; (ii) a tetracycline-inducible expression system; (iii) an AAV promoter (such as p41 or TRE-tight); and (iv) a polynucleotide encoding an AAV capsid protein; (b) a helper plasmid containing one or more helper genes selected from E4, E2a and VA; and (c) a transgene plasmid including a transgene of interest flanked by inverse terminal repeats. The introducing step can be under conditions that permit formation of the recombinant adeno-associated virus. The methods further can include the step of collecting the recombinant adeno-associated virus. The polynucleotide encoding an AAV capsid protein can be a polynucleotide encoding an AAV1, AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP.S capsid protein. The tetracycline-inducible expression system can be a Tet-Off system ora Tet-On system, as described herein. The transgene plasmid of the inventions can further include a promoter operably linked to the transgene of interest. The mammalian cell can be a HEK-293 cell. The transgene of interest can encode a protein that is endogenously expressed in the human inner ear. The transgene of interest can encode solute carrier family 26, member 4 (Pendrin), Otoferlin (OTOF), Stereocilin (STRC), Atonal BHLH Transcription Factor 1 (ATOH1), gap junction protein beta 2 (GJB2), or SRY-Box 2 (Sox2), for example. Other transgenes that can be expressed include, for example, GJB6; WFS1; COCH; EYA4; MYO7A; POU4F3; ACTG1; MY06; REST; NLRP3; COL11A1; TJP2; TBC1D24; SLC26A4; ELMOD3; EPSN; WHRN; IGF1; IGF1R; MYO15A; TMIE; TMC1; TMC2; TMPRSS3; OTOF; CDH23; GIPC3; USH1C; OTOG; TECTA; OTOA; PDCH15; CLDN14; WHRN; ESRRB; MYO3A; HGF, ILDR1, ADCY1; CIB2; MARVELD2; SOX2; SLC26A5; COL4A3; COL4A4; COL4A5; CLPP; PJVK; LRTOMT/COMT2; LOXHD1; TPRM; SYNE4; KCNJ10; PTPRQ; OTOGL; LHFPL5; A1PR2; CABP2; MET; GRXCR2; EPS8; CLIC5; EPS8L2; WBP2; ROR1; CLDN9; USH1G; PKHD1L1; DIAPH3; NDP; USH2A; CLRN1; SANS; HARS1; TRIOBP and other genes. A transgene of interest includes any gene that is desired to be expressed and can fit within an AAV capsid. More than one transgene of interest is possible if the totality of genes are of a combined size that can fit within an AAV capsid.

The viral yield can be at least 1×10¹¹ vg/ml (for example, 1×10¹¹ vg/mL, 2×10¹¹ vg/mL, 3×10¹¹ vg/mL, 4×10¹¹ vg/mL, 5×10¹¹ vg/mL, or greater, such as such as 1×10¹¹ vg/mL to 5×10¹¹ vg/mL, 1×10¹¹ vg/mL to 1×10¹² vg/mL, 1×10¹¹ vg/mL to 5×10¹² vg/mL, or 1×10¹¹ vg/mL to 1×10¹³ vg/mL) as measured using ddPCR of clarified lysate.

In any of the foregoing inventions, the tetracycline-inducible expression system can be a Tet-Off system and the methods can include manufacturing the recombinant adeno-associated virus by exposing the mammalian cell to an inducer of the Tet-Off system (for example, manufacturing in the presence of doxycycline). The methods lead to a higher percentage of full AAV vectors (that is, AAV vectors containing the transgene or nucleic acid to be expressed) than manufacturing the recombinant adeno-associated virus using the same modified rep-cap plasmid in the absence of the inducer of the Tet-Off system (for example, exposing the Tet-Off system to an inducer that can increase the percentage of full AAV vectors by at least 1, 2, 3, 4, 5, 6, 7, 8, 9, 10 or more percentage points, such as from 4% full to 6%, 8%, 10%, 12%, 14% full, or more).

In any of the foregoing inventions, the tetracycline-inducible expression system can be a Tet-On system and the methods can include manufacturing the recombinant adeno-associated virus by maintaining the mammalian cell in the absence of an inducer of the Tet-On system (for example, manufacturing without exposure to doxycycline). The methods lead to a higher percentage of full AAV vectors (that is, AAV vectors containing the transgene or nucleic acid to be expressed) than manufacturing the recombinant adeno-associated virus using the same modified rep-cap plasmid in the presence of the inducer of the Tet-On system (for example, manufacturing AAV vectors without exposing the Tet-On system to an inducer that can increase the percentage of full AAV vectors by at least 1, 2, 3, 4, 5, 6, 7, 8, 9, 10 or more percentage points, such as from 10% full to 12%, 14%, 16%, 18%, 20%, 22% full, or more).

The inventions also provide methods of increasing the yield of a recombinant AAV1-capped adeno-associated virus by at least 2.5-fold (for example, 2.5-fold, 2.7-fold, 3.0-fold, 3.1-fold, 3.5-fold, 4-fold, 4.5-fold, 5-fold, 5.5-fold, 6-fold, 6.5-fold, 7-fold, 8-fold, 9-fold, 10-fold, 11-fold, 12-fold, 13-fold, 14-fold, 15-fold, 20-fold, or more) over a recombinant AAV1-capped adeno-associated virus produced with pAAV-RC1, including the step of substituting a rep-cap plasmid of any one of the foregoing inventions for pAAV-RC1 in manufacturing of the adeno-associated virus. The yield of the recombinant AAV1-capped adeno-associated virus can be increased by at least 7-fold. The yield of the recombinant AAV1-capped adeno-associated virus can be increased by at least 8-fold. The yield of the recombinant AAV1-capped adeno-associated virus can be increased by at least 9-fold. The yield of the recombinant AAV1-capped adeno-associated virus can be increased by at least 10-fold. The rep-cap plasmid can include a polynucleotide sequence of SEQ ID NO: 12. The rep-cap plasmid can include a polynucleotide sequence of SEQ ID NO: 13. The rep-cap plasmid can include a polynucleotide sequence of SEQ ID NO: 22. The tetracycline-inducible expression system can be a Tet-Off system and the methods can be performed in the presence of an inducer of the Tet-Off system. The tetracycline-inducible expression system can be a Tet-On system and the methods can be performed in the absence of an inducer of the Tet-On system. The methods also increase the percentage of full vectors produced.

The inventions further provide methods of increasing both the yield and the percentage of full (that is, AAV vectors containing the transgene or nucleotide sequence of interest to be expressed) recombinant AAV1-capped adeno-associated virus over a recombinant AAV1-capped adeno-associated virus produced with pAAV-RC1, including the step of substituting a rep-cap plasmid of any one of the foregoing inventions that can include a Tet-On system for pAAV-RC1 in manufacturing of the adeno-associated virus. The formation of the recombinant AAV1-capped adeno-associated virus in such methods can be achieved without added inducer (for example, without added doxycycline, meaning that the Tet-On system is not induced). The formation of the recombinant AAV1-capped adeno-associated virus in such methods also can be achieved with added inducer (for example, tetracycline or doxycycline).

The inventions further provide cells comprising the rep-cap plasmids according to the inventions, and cell cultures comprising the cells according to the inventions.

### BRIEF DESCRIPTION OF THE DRAWINGS

FIG. 1 depicts a schematic plasmid map of modified rep-cap plasmid P929.

FIG. 2 depicts a schematic plasmid map of standard rep-cap plasmid P374.

FIG. 3 depicts a schematic plasmid map of modified rep-cap plasmid P2056.

FIG. 4 is a graph showing percentage of full vectors produced using modified rep-cap plasmid P929 in the presence and absence of doxycycline as measured by SEC-MALS.

FIG. 5 depicts a schematic plasmid map of rep-cap plasmid P2057.

FIG. 6 depicts a schematic plasmid map of rep-cap plasmid P2058.

FIG. 7 depicts a schematic plasmid map of rep-cap plasmid P2059.

FIGS. 8A and 8B depict data from production of AAV1 containing a GJB2 transgene using an RC1 p5AS plasmid or P929. FIG. 8A depicts genomic titer and FIG. 8B depicts capsid titer.

FIGS. 9A to 9C depict production of AAV1 containing an enhanced green fluorescent protein (eGFP) transgene using plasmids P374, P929, P2057, P2058 or P2059. FIG. 9A compares capsid titer for each of the five plasmids. FIG. 9B compares genomic titer using droplet digital PCR (ddPCR) for each of the five plasmids. FIG. 9C compares estimated genomic titer based on percent full capsids for each of the five plasmids.

FIGS. 10A to 10B depict production of AAV1 containing an eGFP transgene using plasmids P374, P929, P2057, P2058 or P2059, and employ mass photometry to assess capsid fullness achieved by each of the five plasmids. FIG. 10A sets forth the percentage of full capsids. FIG. 10B sets forth the percentage of partially-filled capsids.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

As used herein, the terms “AAV” and “adeno-associated virus” refer to a Dependoparvovirus within the Parvoviridae genus of viruses. AAV can refer to an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene.

As used herein, the term “rAAV” refers to a recombinant AAV. Recombinant AAV refers to an AAV genome in which part or all of the rep and cap genes have been replaced with heterologous sequences.

As used herein, the term “polynucleotide encoding an AAV capsid protein” refers to the nucleic acid sequences that encode capsid proteins that form, or contribute to the formation of, the capsid, or protein shell, of the virus. In the case of AAV, the polynucleotide encoding an AAV capsid protein encodes capsid proteins VP1, VP2, and VP3.

As used herein, “administration” refers to providing or giving a subject a therapeutic agent (for example, a rAAV1 vector produced using a modified rep-cap plasmid described herein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (for example, epithelial tissue, neural tissue, connective tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in Table 1, below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky.

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (for example, a substitution of Ser for Thr or Lys for Arg).

As used herein, the term “endogenous” refers to a molecule (for example, a polypeptide, nucleic acid, or cofactor) that is found naturally in a particular organism (for example, a human) or in a particular location within an organism (for example, an organ, a tissue, or a cell, such as a human cell, for example, a human cochlear supporting cell).

As used herein, the term “express” refers to one or more of the following events: (1) production of an RNA template from a DNA sequence (for example, by transcription); (2) processing of an RNA transcript (for example, by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (for example, a polypeptide, nucleic acid, or cofactor) that is not found naturally in a particular organism (for example, a human) or in a particular location within an organism (for example, an organ, a tissue, or a cell, such as a human cell, for example, a human cochlear supporting cell). Exogenous materials include those that are provided from an external source to an organism or to cultured matter extracted therefrom.

As used herein, the term “heterologous” refers to a combination of elements that is not naturally occurring. For example, a heterologous transgene refers to a transgene that is not naturally expressed by the promoter to which it is operably linked.

As used herein, the term “target host cell” refers to a cell that has been transformed, or is capable of being transformed, with a nucleic acid sequence and thereby expresses a gene of interest, and preferably will be from a cell line. The term includes the progeny of the parent cell, whether or not the progeny is identical in morphology or in genetic make-up to the original parent cell, so long as the gene of interest is present.

Target host cells that are suitable for use with the inventions can be readily selected by those of skill in the art. In some embodiments the cell line is a eukaryotic cell line such as a yeast cell line, insect cell line (for example, Sf9 and Sf21 cells) or mammalian cell lines. Preferred mammalian cells include primate cells (including human), canine cells and rodent cells. Cells can be primary cells or immortalized cells. Suitable cells can be selected from Vero cells, COS cells, HEK 293 cells, Hela cells, CHO cells, BHK cells, MDCK cells, amniotic cells (human), embryonic cells, cell lines transfected with viral genes, for example, AD5 E1, including but not limited to an immortalized human retinal cell transfected with an adenovirus gene, for example, a PER. C6 cell, or an NSO cell. In some embodiments, the cell is a Chinese hamster ovary (CHO) cell line. Some examples of CHO cells include, but are not limited to, CHO-ori, CHO-K1, CHO-s, CHO-DHB11, CHO-DXB11, CHO-K1SV, and mutants and variants thereof. In other embodiments, the cell is a HEK293 cell. Some examples of HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof.

As used herein, the term “naturally occurring” refers to materials which are found in nature or a form of the material that is found in nature.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (for example, an intervening non-coding polynucleotide) or may be operably linked to one another with no intervening nucleotides present.

As used herein, the term “pAAV-RC1” refers to a vector containing a polynucleotide encoding an AAV2 rep protein and a polynucleotide encoding an AAV1 capsid protein that does not include a tetracycline-inducible expression system. Such a vector is produced by Cell Biolabs, Inc. (cellbiolabs.com/sites/default/files/VPK-421-aav-rc1-vector.pdf).

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (for example, bacterial plasmids having a bacterial origin of replication and episomal mammalian plasmids). Other vectors (for example, non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (for example, adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (that is, the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene. AAV promoters include, but are not limited to, preferred promoters p41 and TRE-tight. Other AAV promoters that can be used include the p40 promoter.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (for example, BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, for example, a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (for example, a human) without excessive toxicity, irritation, allergic response, and other problem complications commensurate with a reasonable benefit/risk ratio.

As used herein, the term “polynucleotide encoding an AAV rep protein” refers to the nucleic acid sequences that encode the non-structural proteins (that is, rep78, rep68, rep52, and rep40) required for the replication and production of virus.

As used herein, the term “rep-cap plasmid” refers to a plasmid that provides the viral rep and cap gene functions. This plasmid can be useful for the production of AAVs from rAAV genomes lacking functional rep and/or the capsid gene sequences.

As used herein, the term “subject” refers to any organism to which a composition in accordance with the invention may be administered, for example, for experimental, diagnostic, prophylactic, and/or therapeutic purposes. Typical subjects include any animal (for example, mammals such as mice, rats, rabbits, non-human primates, and humans). A subject may seek and be in need of treatment, require treatment, be receiving treatment, be receiving treatment in the future, and be a human and animal who is under care by a trained professional for a particular disease and condition. Preferably, the subject is a human.

As used herein, the terms “transcription regulatory element” and “regulatory sequence” refer to a polynucleotide that controls, at least in part, the transcription of a gene of interest. Transcription regulatory elements may include promoters, enhancers, and other polynucleotides (for example, polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, for example, electroporation, lipofection, calcium phosphate precipitation, DEAE-dextran transfection, Nucleofection, squeeze-poration, sonoporation, optical transfection, magnetofection, impalefection and the like.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, the term “transgene” refers to a recombinant nucleic acid (for example, DNA) encoding a gene product, such as a peptide, protein, or RNA (for example, a protein-encoding mRNA or an inhibitory RNA, such as a miRNA, shRNA, or the like). In addition to the coding region for the gene product, the transgene may include, or be operably linked to, one or more elements to facilitate or enhance expression, such as a promoter, enhancer(s), destabilizing domain(s), response element(s), reporter element(s), insulator element(s), polyadenylation signal(s), and/or other functional elements. According to the current inventions, any known suitable promoter, enhancer(s), destabilizing domain(s), response element(s), reporter element(s), insulator element(s), polyadenylation signal(s), and/or other functional elements can be utilized. Examples of transgenes are identified herein.

As used herein, “treatment” and “treating,” in reference to a disease or condition, refer to an approach for obtaining beneficial or desired results, for example, clinical results. Beneficial or desired results can include, but are not limited to, alleviation or amelioration of one or more symptoms or conditions; diminishment of extent of disease or condition; stabilized (that is, not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, for example, a DNA vector, such as a plasmid, cosmid, or artificial chromosome, an RNA vector, a virus, or any other suitable replicon (for example, viral vector). A variety of vectors have been developed for the delivery of polynucleotides encoding exogenous proteins into a prokaryotic or eukaryotic cell. Examples of such expression vectors are described in, for example, Gellissen, Production of Recombinant Proteins: Novel Microbial and Eukaryotic Expression Systems (John Wiley & Sons, Marblehead, MA, 2006). Expression vectors suitable for use with the compositions and methods described herein contain a polynucleotide sequence as well as, for example, additional sequence elements used for the expression of proteins. Certain vectors that can be used for the expression of a viral capsid protein as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, for example, 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “viral capsid protein” refers to a capsid protein composing a proteinaceous shell. Such a proteinaceous shell is generally composed of one or more viral capsid proteins and when assembled is capable of being loaded with one or more polynucleotide molecules. A viral capsid protein described herein may, for example, be a viral protein (VP) 1, VP2, and VP3. Further, a viral capsid protein described herein may refer to a viral capsid protein from an AAV1.

As used herein, the term “VP1” refers to a capsid protein that is a component of an AAV capsid, such as AAV1. The AAV1 VP1 protein has the sequence of SEQ ID NO: 9. As used herein, a VP1 may possess a surface binding site that interacts with one or more molecules on the surface of a cell to initiate the process of cell entry (for example, endocytic entry and receptor-mediated fusion). As used herein, a VP1 may self-assemble into a structure consisting of VP1, VP2, and/or VP3 molecules. VP1 may exhibit self-binding properties and self-assemble around the exterior of a respective VP1-containing capsid.

As used herein, the term “VP2” refers to a capsid protein that is a component of an AAV capsid, such as AAV1. The AAV1 VP2 protein has the sequence of SEQ ID NO: 14. As used herein, a VP2 may facilitate capsid entry into a host cell, for example, by mediating associations with and exit from the endoplasmic reticulum of a host cell and by facilitating the entry of a nucleic acid molecule into a host cell nucleus. As used herein, a VP2 may self-assemble into a structure consisting of VP1, VP2, and/or VP3 molecules. VP2 may self-assemble within the interior of a respective VP2-containing capsid.

As used herein, the term “VP3” refers to a capsid protein that is a component of an AAV capsid, such as AAV1. The AAV1 VP3 protein has the sequence of SEQ ID NO: 15. As used herein, a VP3 may facilitate capsid entry into a host cell, for example, by mediating associations with and exit from the endoplasmic reticulum of a host cell and by facilitating the entry of a nucleic acid molecule into a host cell nucleus. As used herein, a VP3 may self-assemble into a structure consisting of VP1, VP2, and/or VP3 molecules. VP3 may self-assemble within the interior of a respective VP3-containing capsid.

As used herein, the term “wild-type” refers to a genotype with the highest frequency for a particular gene in a given organism.

### DESCRIPTION

Described herein are compositions and methods for producing AAV vectors (for example, AAV1 vectors). The present inventions provide modified rep-cap plasmids containing a tetracycline-inducible expression system (for example, a Tet-Off or a Tet-On system) positioned between a polynucleotide encoding an AAV rep protein and a polynucleotide encoding an AAV capsid protein (for example, an AAV1 capsid protein). The polynucleotide encoding an AAV capsid protein (for example, AAV1 capsid protein) can encode AAV capsid proteins VP1, VP2, and VP3. In addition, the inventions provide methods of producing AAV vectors (for example, AAV1 vectors) using the modified rep-cap plasmid (for example, by transducing a cell with the modified rep-cap plasmid, a helper plasmid, and a transgene plasmid). The methods can include turning the tetracycline-inducible expression system “off” (for example, producing AAV vectors using a modified rep-cap plasmid that contains a Tet-Off system in the presence of an inducer of the Tet-Off system, or producing AAV vectors using a modified rep-cap plasmid that contains a Tet-On system in the absence of an inducer of the Tet-On system). The compositions and methods described herein can be used to increase AAV production (for example, AAV1 production) compared to approaches using unmodified rep-cap plasmids (for example, rep-cap plasmids containing the same polynucleotides encoding rep and capsid proteins but lacking the tetracycline-inducible expression system). In addition, the compositions and methods described herein can be used to increase the percent of full AAV vectors produced (for example, AAV vectors containing a transgene or polynucleotide to be expressed) compared to approaches using the same modified rep-cap plasmids in which the tetracycline-inducible expression system is turned “on.” AAV vectors (for example, AAV1 vectors) produced using the methods described herein can contain a transgene of interest and can be used in gene therapy applications.

### Modified Rep-Cap Plasmids

Production of AAV vectors requires three plasmids: 1) a rep-cap plasmid, which contains the AAV structural and packaging genes; 2) a helper plasmid, which contains genes that encode proteins necessary for viral replication; and 3) a transgene plasmid (also referred to as a transfer plasmid), which contains a transgene to be expressed by the AAV vector. The rep-cap plasmid contains two genes, the replication (rep) gene and the capsid (cap) gene. The rep gene encodes four proteins involved in viral genome replication and packaging: Rep78, Rep68, Rep52, and Rep40. The cap gene encodes three AAV viral capsid proteins, VP1, VP2, and VP3, which together form the outer capsid shell that protects the viral genome. Alternative splicing and differing translational start sites generate the three VP proteins. The cap gene also encodes the Assembly-Activating Protein (AAP) and the Membrane Associated Accessory Protein (MAAP) from an alternative open reading frame. AAP is thought to provide a scaffolding function for capsid assembly.

The present inventions are based, in part, on the discovery of modifications to the rep-cap plasmid that can result in increased AAV production, such as AAV1 (for example, when the modified rep-cap plasmid is introduced into a cell with a helper plasmid and a transgene plasmid). These modifications can include introduction of a tetracycline-inducible expression system between a polynucleotide encoding an AAV rep protein and a polynucleotide encoding an AAV1 capsid protein. Producing greater amounts of AAV vector, such as AAV1, is desirable because it facilitates manufacturing and can reduce the amount of concentration needed to achieve a dose effective for gene therapy applications in a reasonable volume. The present inventors also made the surprising discovery that a higher percentage of full vectors could be produced using the modified rep-cap plasmids by manufacturing AAV vectors in conditions that would cause the tetracycline-inducible expression system to be turned “off” (for example, producing AAV vectors using a modified rep-cap plasmid that contains a Tet-Off system in the presence of an inducer of the Tet-Off system, or producing AAV vectors using a modified rep-cap plasmid that contains a Tet-On system in the absence of an inducer of the Tet-On system). Producing a higher percentage of full vectors is desirable because it means that more of the resulting product can be used for therapy. The compositions and methods described herein can, thus, be used to manufacture AAV1 vector for use in gene therapy (for example, a therapy that involves expressing a transgene in a cell that can be transduced by AAV1 vectors).

The modified rep-cap plasmids described herein can include a polynucleotide encoding an AAV rep protein and a polynucleotide encoding an AAV cap protein, such as an AAV1 cap protein. The plasmids can contain any of the polynucleotide sequences disclosed or identified herein, as well other existing polynucleotides, including regulatory sequences, that are useful for making the rep-cap plasmids described herein.

The polynucleotide encoding an AAV rep protein can be a polynucleotide encoding an AAV2 rep protein, for example. The polynucleotide encoding an AAV rep protein can encode both Rep78 and Rep52. The Rep78 protein encoded by the polynucleotide encoding an AAV rep protein (for example, an AAV2 rep protein) can have the amino acid sequence of SEQ ID NO: 1, provided below.

(SEQ ID NO: 1)
MPGFYEIVIKVPSDLDEHLPGISDSFVNWVAEKEWELPPDSDMDLNLIE
QAPLTVAEKLQRDFLTEWRRVSKAPEALFFVQFEKGESYFHMHVLVETT
GVKSMVLGRFLSQIREKLIQRIYRGIEPTLPNWFAVTKTRNGAGGGNKV
VDECYIPNYLLPKTQPELQWAWTNMEQYLSACLNLTERKRLVAQHLTHV
SQTQEQNKENQNPNSDAPVIRSKTSARYMELVGWLVDKGITSEKQWIQE
DQASYISFNAASNSRSQIKAALDNAGKIMSLTKTAPDYLVGQQPVEDIS
SNRIYKILELNGYDPQYAASVFLGWATKKFGKRNTIWLFGPATTGKTNI
AEAIAHTVPFYGCVNWTNENFPFNDCVDKMVIWWEEGKMTAKWVESAKA
ILGGSKVRVDQKCKSSAQIDPTPVIVTSNTNMCAVIDGNSTTFEHQQPL
QDRMFKFELTRRLDHDFGKVTKQEVKDFFRWAKDHVVEVEHEFYVKKGG
AKKRPAPSDADISEPKRVRESVAQPSTSDAEASINYADRYQNKCSRHVG
MNLMLFPCRQCERLNQNSNICFTHGVKDCLECFPVSESQPVSVVKKAYQ
KLCYIHHIMGKVPDACTACDLVNVDLDDCVSEQ

The Rep52 protein encoded by the polynucleotide encoding an AAV rep protein (for example, an AAV2 rep protein) can have the amino acid sequence of SEQ ID NO: 2, provided below.

(SEQ ID NO: 2)
MELVGWLVDKGITSEKQWIQEDQASYISFNAASNSRSQIKAALDNAGKI
MSLTKTAPDYLVGQQPVEDISSNRIYKILELNGYDPQYAASVFLGWATK
KFGKRNTIWLFGPATTGKTNIAEAIAHTVPFYGCVNWTNENFPFNDCVD
KMVIWWEEGKMTAKVVESAKAILGGSKVRVDQKCKSSAQIDPTPVIVTS
NTNMCAVIDGNSTTFEHQQPLQDRMFKFELTRRLDHDFGKVTKQEVKDF
FRWAKDHVVEVEHEFYVKKGGAKKRPAPSDADISEPKRVRESVAQPSTS
DAEASINYADRYQNKCSRHVGMNLMLFPCRQCERLNQNSNICFTHGVKD
CLECFPVSESQPVSVVKKAYQKLCYIHHIMGKVPDACTACDLVNVDLDD
CVSEQ

The polynucleotide encoding an AAV rep protein included in the rep-cap plasmids described herein can be a polynucleotide that can have the sequence of SEQ ID NO: 3, provided below.

(SEQ ID NO: 3)
ATGCCGGGGTTTTACGAGATTGTGATTAAGGTCCCCAGCGACCTTGA
CGAGCATCTGCCCGGCATTTCTGACAGCTTTGTGAACTGGGTGGCCGAG
AAGGAATGGGAGTTGCCGCCAGATTCTGACATGGATCTGAATCTGATTG
AGCAGGCACCCCTGACCGTGGCCGAGAAGCTGCAGCGCGACTTTCTGAC
GGAATGGCGCCGTGTGAGTAAGGCCCCGGAGGCTCTTTTCTTTGTGCAA
TTTGAGAAGGGAGAGAGCTACTTCCACATGCACGTGCTCGTGGAAACCA
CCGGGGTGAAATCCATGGTTTTGGGACGTTTCCTGAGTCAGATTCGCGA
AAAACTGATTCAGAGAATTTACCGCGGGATCGAGCCGACTTTGCCAAAC
TGGTTCGCGGTCACAAAGACCAGAAATGGCGCCGGAGGCGGGAACAAGG
TGGTGGATGAGTGCTACATCCCCAATTACTTGCTCCCCAAAACCCAGCC
TGAGCTCCAGTGGGCGTGGACTAATATGGAACAGTATTTAAGCGCCTGT
TTGAATCTCACGGAGCGTAAACGGTTGGTGGCGCAGCATCTGACGCACG
TGTCGCAGACGCAGGAGCAGAACAAAGAGAATCAGAATCCCAATTCTGA
TGCGCCGGTGATCAGATCAAAAACTTCAGCCAGGTACATGGAGCTGGTC
GGGTGGCTCGTGGACAAGGGGATTACCTCGGAGAAGCAGTGGATCCAGG
AGGACCAGGCCTCATACATCTCCTTCAATGCGGCCTCCAACTCGCGGTC
CCAAATCAAGGCTGCCTTGGACAATGCGGGAAAGATTATGAGCCTGACT
AAAACCGCCCCCGACTACCTGGTGGGCCAGCAGCCCGTGGAGGACATTT
CCAGCAATCGGATTTATAAAATTTTGGAACTAAACGGGTACGATCCCCA
ATATGCGGCTTCCGTCTTTCTGGGATGGGCCACGAAAAAGTTCGGCAAG
AGGAACACCATCTGGCTGTTTGGGCCTGCAACTACCGGGAAGACCAACA
TCGCGGAGGCCATAGCCCACACTGTGCCCTTCTACGGGTGCGTAAACTG
GACCAATGAGAACTTTCCCTTCAACGACTGTGTCGACAAGATGGTGATC
TGGTGGGAGGAGGGGAAGATGACCGCCAAGGTCGTGGAGTCGGCCAAAG
CCATTCTCGGAGGAAGCAAGGTGCGCGTGGACCAGAAATGCAAGTCCTC
GGCCCAGATAGACCCGACTCCCGTGATCGTCACCTCCAACACCAACATG
TGCGCCGTGATTGACGGGAACTCAACGACCTTCGAACACCAGCAGCCGT
TGCAAGACCGGATGTTCAAATTTGAACTCACCCGCCGTCTGGATCATGA
CTTTGGGAAGGTCACCAAGCAGGAAGTCAAAGACTTTTTCCGGTGGGCA
AAGGATCACGTGGTTGAGGTGGAGCATGAATTCTACGTCAAAAAGGGTG
GAGCCAAGAAAAGACCCGCCCCCAGTGACGCAGATATAAGTGAGCCCAA
ACGGGTGCGCGAGTCAGTTGCGCAGCCATCGACGTCAGACGCGGAAGCT
TCGATCAACTACGCGGACAGGTACCAAAACAAATGTTCTCGTCACGTGG
GCATGAATCTGATGCTGTTTCCCTGCAGACAATGCGAGAGACTGAATCA
GAATTCAAATATCTGCTTCACTCACGGTGTCAAAGACTGTTTAGAGTGC
TTTCCCGTGTCAGAATCTCAACCCGTTTCTGTCGTCAAAAAGGCGTATC
AGAAACTGTGCTACATTCATCACATCATGGGAAAGGTGCCAGACGCTTG
CACTGCTTGCGACCTGGTCAATGTGGACTTGGATGACTGTGTTTCTGAA
CAA

The modified rep-cap plasmids described herein also can include a tetracycline-inducible expression system. The tetracycline-inducible expression system can be positioned 3′ of the polynucleotide encoding an AAV rep protein within the modified rep-cap plasmid.

The tetracycline-inducible expression system can be a tetracycline off (Tet-Off) system. The Tet-Off system can include a tetracycline-controlled transactivator (ITA) and one or more (for example, 1, 2, 3, 4, 5, 6, 7, 8, 9, 10, 11, 12, or more) tetracycline operator (tetO) sequences. The tTA can contain a tetracycline repressor (tetR)-derived DNA binding domain and a transcriptional activation domain. The tTA can be a fusion of a tetR and the C-terminal domain of VP16 (virion protein 16), an essential transcriptional activation domain from HSV (herpes simplex virus). The Tet-Off system can contain 7 tetO sequences. The Tet-Off system can contain 8 tetO sequences. The one or more (for example, 1, 2, 3, 4, 5, 6, 7, 8, 9, 10, 11, 12, or more) tetO sequences can be positioned directly next to one another (for example, joined without any intervening sequence between the tetO sequences, for example, the 3′ end of a first tetO sequence is positioned directly before the 5′ end of a second tetO sequence) or can be joined by a nucleic acid linker (for example, a nucleic acid linker can be positioned between each tetO sequence included in the Tet-Off system or between at least two of the tetO sequences in the Tet-Off system). According to the current inventions, where one or more tetO sequences can be joined by a linker, each linker can contain 1-20 or more nucleotides (for example, 1, 2, 3, 4, 5, 6, 7, 8, 9, 10, 11, 12, 13, 14, 15, 16, 17, 18, 19, 20 or more nucleotides) and does not disrupt the function of the tetO sequences. Each tetO sequence in the Tet-Off system can be separated from the next tetO sequence by a linker containing 20 nucleotides or more. Each tetO sequence in the Tet-Off system can be separated from the next tetO sequence by a linker containing 18 nucleotides. Within the Tet-Off system, the polynucleotide sequence encoding the tTA can be positioned 5′ of the one or more tetO sequences. The tetracycline-inducible expression system can include a polyadenylation (polyA) signal sequence positioned 3′ of the polynucleotide sequence encoding the tTA and 5′ of the polynucleotide sequence encoding the tetO sequences (that is, the polyA signal sequence can be positioned between the tTA sequence and the tetO sequences in the modified rep-cap plasmid).

The tTA can include a protein having at least 85% sequence identity (for example, 85%, 86%, 87%, 88%, 89%, 90%, 91%, 92%, 93%, 94%, 95%, 96%, 97%, 98%, 99%, or more, sequence identity) to SEQ ID NO: 5 and which retains function as a tTA (that is, excludes proteins having a sequence that functions as a rtTA). The tTA can include a protein having an amino acid sequence that contains one or more conservative amino acid substitutions relative to SEQ ID NO: 5 (for example, 1, 2, 3, 4, 5, 6, 7, 8, 9, 10, 11, 12, 13, 14, 15, 16, 17, 18, 19, 20 or more conservative amino acid substitutions), provided that the tTA analog encoded retains the ability to bind to the tetO sequences and promote expression in the absence of an effector. The inventions provide that no more than 10% of the amino acids in the tTA protein can be replaced with conservative amino acid substitutions. The polynucleotide sequence that encodes a tTA protein can be any polynucleotide sequence that, by redundancy of the genetic code, encodes SEQ ID NO: 5. The tTA can have the amino acid sequence of SEQ ID NO: 5, provided below.

(SEQ ID NO: 5)
MSRLDKSKVINGALELLNEVGIEGLTTRKLAQKLGVEQPTLYWHVKNKR
ALLDALAIEMLDRHHTHFCPLEGESWQDFLRNNAKSFRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGGPADALDDFDLDMLPADALDDFDLDMLPADALDDFDLDM
LPG

The tTA protein of SEQ ID NO: 5 can be encoded by a polynucleotide having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 6. The tTA protein of SEQ ID NO: 5 can be encoded by the polynucleotide sequence of SEQ ID NO: 6, provided below.

(SEQ ID NO: 6)
ATGAGTCGGCTGGATAAATCTAAAGTCATAAACGGCGCTCTGGAATT
ACTCAATGAAGTCGGTATCGAAGGCCTGACGACAAGGAAACTCGCTCAA
AAGCTGGGAGTTGAGCAGCCTACCCTGTACTGGCACGTGAAGAACAAGC
GGGCCCTGCTCGATGCCCTGGCCATCGAGATGCTGGACAGGCATCATAC
CCACTTCTGCCCCCTGGAAGGCGAGTCATGGCAAGACTTTCTGCGGAAC
AACGCCAAGTCATTCCGCTGTGCTCTCCTCTCACATCGCGACGGGGCTA
AAGTGCATCTCGGCACCCGCCCAACAGAGAAACAGTACGAAACCCTGGA
AAATCAGCTCGCGTTCCTGTGTCAGCAAGGCTTCTCCCTGGAGAACGCA
CTGTACGCTCTGTCCGCCGTGGGCCACTTTACACTGGGCTGCGTATTGG
AGGAACAGGAGCATCAAGTAGCAAAAGAGGAAAGAGAGACACCTACCAC
CGATTCTATGCCCCCACTTCTGAGACAAGCAATTGAGCTGTTCGACCGG
CAGGGAGCCGAACCTGCCTTCCTTTTCGGCCTGGAACTAATCATATGTG
GCCTGGAGAAACAGCTAAAGTGCGAAAGCGGCGGGCCGGCCGACGCCCT
TGACGATTTTGACTTAGACATGCTCCCAGCCGATGCCCTTGACGACTTT
GACCTTGATATGCTGCCTGCTGACGCTCTTGACGATTTTGACCTTGACA
TGCTCCCCGGGTAA

The tetracycline-inducible expression system can be a tetracycline on (Tet-On) system. The Tet-On system can include a reverse tetracycline-controlled transactivator (rtTA) and one or more (for example, 1, 2, 3, 4, 5, 6, 7, 8, 9, 10, 11, 12, or more) tetracycline operator (tetO) sequences. The rtTA can contain a variant of a tetracycline repressor (tetR)-derived DNA binding domain that can bind tetO only in the presence of an effector (called a reverse tetR). The inventions also provide that rtTA can be a fusion of the reverse tetR and the C-terminal domain of VP16 (virion protein 16), an essential transcriptional activation domain from HSV (herpes simplex virus). The Tet-On system can contain 7 tetO sequences. The Tet-On system can contain 8 tetO sequences. The one or more (for example, 1, 2, 3, 4, 5, 6, 7, 8, 9, 10, 11, 12, or more) tetO sequences can be positioned directly next to one another (for example, joined without any intervening sequence between the tetO sequences, for example, the 3′ end of a first tetO sequence is positioned directly before the 5′ end of a second tetO sequence) or can be joined by a nucleic acid linker (for example, a nucleic acid linker can be positioned between each tetO sequence included in the Tet-On system or between at least two of the tetO sequences in the Tet-On system). According to the current inventions, where one or more tetO sequences can be joined by a linker, each linker can contain 1-20 nucleotides (for example, 1, 2, 3, 4, 5, 6, 7, 8, 9, 10, 11, 12, 13, 14, 15, 16, 17, 18, 19, or 20 nucleotides) and does not disrupt the function of the tetO sequences. Each tetO sequence in the Tet-On system can be separated from the next tetO sequence by a linker containing 20 nucleotides. Each tetO sequence in the Tet-On system can be separated from the next tetO sequence by a linker containing 18 nucleotides. Within the Tet-On system, the polynucleotide sequence encoding the rtTA can be positioned 5′ of the one or more tetO sequences. The tetracycline-inducible expression system can include a polyadenylation (polyA) signal sequence positioned 3′ of the polynucleotide sequence encoding the rtTA and 5′ of the polynucleotide sequence encoding the tetO sequences (that is, the polyA signal sequence can be positioned between the rtTA sequence and the tetO sequences in the modified rep-cap plasmid). The rtTA can include a protein having at least 85% sequence identity (for example, 85%, 86%, 87%, 88%, 89%, 90%, 91%, 92%, 93%, 94%, 95%, 96%, 97%, 98%, 99%, or more, sequence identity) to SEQ ID NO: 20, and which can retain function as a rtTA (that is, excludes proteins having a sequence that functions as a tTA). The rtTA can include a protein having an amino acid sequence that can contain one or more conservative amino acid substitutions relative to SEQ ID NO: 20 (for example, 1, 2, 3, 4, 5, 6, 7, 8, 9, 10, 11, 12, 13, 14, 15, 16, 17, 18, 19, 20 or more conservative amino acid substitutions), provided that the rtTA analog encoded can retain the ability to bind to the tetO sequences and promote expression in the presence of an effector. The inventions provide that no more than 10% of the amino acids in the rtTA protein can be replaced with conservative amino acid substitutions. The polynucleotide sequence that encodes a rtTA protein can be any polynucleotide sequence that, by redundancy of the genetic code, can encode SEQ ID NO: 20. The rtTA can have the amino acid sequence of SEQ ID NO: 20, provided below.

(SEQ ID NO: 20)
MSRLDKSKVINSALELLNGVGIEGLTTRKLAQKLGVEQPTLYWHVKNKR
ALLDALPIEMLDRHHTHSCPLEGESWQDFLRNNAKSYRCALLSHRDGAK
VHLGTRPTEKQYETLENQLAFLCQQGFSLENALYALSAVGHFTLGCVLE
EQEHQVAKEERETPTTDSMPPLLKQAIELFDRQGAEPAFLFGLELIICG
LEKQLKCESGGPTDALDDFDLDMLPADALDDFDLDMLPADALDDFDLDM
LPG

The rtTA protein of SEQ ID NO: 20 can be encoded by a polynucleotide having at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 21. The rtTA protein of SEQ ID NO: 20 can be encoded by the polynucleotide sequence of SEQ ID NO: 21, provided below.

(SEQ ID NO: 21)
ATGAGTCGGCTGGATAAATCTAAAGTCATAAACTCTGCTCTGGAATTA
CTCAATGGAGTCGGTATCGAAGGCCTGACGACAAGGAAACTCGCTCAAA
AGCTGGGAGTTGAGCAGCCTACCCTGTACTGGCACGTGAAGAACAAGCG
GGCCCTGCTCGATGCCCTGCCAATCGAGATGCTGGACAGGCATCATACC
CACTCCTGCCCCCTGGAAGGCGAGTCATGGCAAGACTTTCTGCGGAACA
ACGCCAAGTCATACCGCTGTGCTCTCCTCTCACATCGCGACGGGGCTAA
AGTGCATCTCGGCACCCGCCCAACAGAGAAACAGTACGAAACCCTGGAA
AATCAGCTCGCGTTCCTGTGTCAGCAAGGCTTCTCCCTGGAGAACGCAC
TGTACGCTCTGTCCGCCGTGGGCCACTTTACACTGGGCTGCGTATTGGA
GGAACAGGAGCATCAAGTAGCAAAAGAGGAAAGAGAGACACCTACCACC
GATTCTATGCCCCCACTTCTGAAACAAGCAATTGAGCTGTTCGACCGGC
AGGGAGCCGAACCTGCCTTCCTTTTCGGCCTGGAACTAATCATATGTGG
CCTGGAGAAACAGCTAAAGTGCGAAAGCGGCGGGCCGACCGACGCCCTT
GACGATTTTGACTTAGACATGCTCCCAGCCGATGCCCTTGACGACTTTG
ACCTTGATATGCTGCCTGCTGACGCTCTTGACGATTTTGACCTTGACAT
GCTCCCCGGGTAA

Each of the one or more tetO sequences included in the tetracycline-inducible expression system (for example, Tet-Off or Tet-On system) in the modified rep-cap plasmids described herein can have the sequence:

TCCCTATCAGTGATAGAGA (SEQ ID NO: 4). The tetracycline-inducible expression system (for example, Tet-Off or Tet-On system) can include eight tetO sequences and the polynucleotide containing the eight tetO sequences can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 7. The polynucleotide containing the 8 tetO sequences can have at least 80% sequence identity (for example, 80%, 81%, 82%, 83%, 84%, 85%, 86%, 87%, 88%, 89%, 90%, 91%, 92%, 93%, 94%, 95%, 96%, 97%, 98%, 99%, or more, sequence identity) to the sequence of SEQ ID NO: 7 apart from the eight tetO sequences, each of which can have the sequence of SEQ ID NO: 4. The portion of the tetracycline-inducible expression system containing the eight tetO sequences can have the sequence of SEQ ID NO: 7, provided below.

(SEQ ID NO: 7)
TCCCTATCAGTGATAGAGAAGTCGACACGTCTCGAGCTCCCTATCAG
TGATAGAGAAGGTACGTCTAGAACGTCTCCCTATCAGTGATAGAGAAGT
CGACACGTCTCGAGCTCCCTATCAGTGATAGAGAAGGTACGTCTAGAAC
GTCTCCCTATCAGTGATAGAGAAGTCGACACGTCTCGAGCTCCCTATCA
GTGATAGAGAAGGTACGTCTAGAACGTCTCCCTATCAGTGATAGAGAAG
TCGACACGTCTCGAGCTCCCTATCAGTGATAGAGA

The modified rep-cap plasmids described herein can include an AAV promoter positioned between the tetracycline-inducible expression system and the polynucleotide encoding an AAV1 capsid protein. The AAV promoter can be an AAV p41 promoter. The p41 promoter can have the sequence of SEQ ID NO: 8, provided below.

(SEQ ID NO: 8)
TGTTCAAATTTGAACTGACTAAGCGGCTCCCGCCAGATTTTGGCAAG
ATTACTAAGCAGGAAGTCAAGGACTTTTTTGCTTGGGCAAAGGTCAATC
AGGTGCCGGTGACTCACGAGTTTAAAGTTCCCAGGGAATTGGCGGGAAC
TAAAGGGGCGGAGAAATCTCTAAAACGCCCACTGGGTGACGTCACCAAT
ACTAGCTATAAAAGTCTGGAGAAGCGGGCCAGGCTCTCATTTGTTCCCG
AGACGCCTCGCAGTTCAGACGTGACTGTTGATCCCGCTCCTCTGCGACC
GCT

The modified rep-cap plasmids described herein can be used to produce AAV vectors containing an AAV1 capsid.

The AAV1 capsid protein VP1 can have the amino acid sequence of SEQ ID NO: 9.

(SEQ ID NO: 9)
MAADGYLPDWLEDNLSEGIREWWDLKPGAPKPKANQQKQDDGRGLVL
PGYKYLGPFNGLDKGEPVNAADAAALEHDKAYDQQLKAGDNPYLRYNHA
DAEFQERLQEDTSFGGNLGRAVFQAKKRVLEPLGLVEEGAKTAPGKKRP
VEQSPQEPDSSSGIGKTGQQPAKKRLNFGQTGDSESVPDPQPLGEPPAT
PAAVGPTTMASGGGAPMADNNEGADGVGNASGNWHCDSTWLGDRVITTS
TRTWALPTYNNHLYKQISSASTGASNDNHYFGYSTPWGYFDFNRFHCHF
SPRDWQRLINNNWGFRPKRLNFKLFNIQVKEVTTNDGVTTIANNLTSTV
QVFSDSEYQLPYVLGSAHQGCLPPFPADVFMIPQYGYLTLNNGSQAVGR
SSFYCLEYFPSQMLRTGNNFTFSYTFEEVPFHSSYAHSQSLDRLMNPLI
DQYLYYLNRTQNQSGSAQNKDLLFSRGSPAGMSVQPKNWLPGPCYRQQR
VSKTKTDNNNSNFTWTGASKYNLNGRESIINPGTAMASHKDDEDKFFPM
SGVMIFGKESAGASNTALDNVMITDEEEIKATNPVATERFGTVAVNFQS
SSTDPATGDVHAMGALPGMVWQDRDVYLQGPIWAKIPHTDGHFHPSPLM
GGFGLKNPPPQILIKNTPVPANPPAEFSATKFASFITQYSTGQVSVEIE
WELQKENSKRWNPEVQYTSNYAKSANVDFTVDNNGLYTEPRPIGTRYLT
RPL

The AAV1 capsid protein VP2 can have the amino acid sequence of SEQ ID NO: 14.

(SEQ ID NO: 14)
TAPGKKRPVEQSPQEPDSSSGIGKTGQQPAKKRLNFGQTGDSESVPDP
QPLGEPPATPAAVGPTTMASGGGAPMADNNEGADGVGNASGNWHCDSTW
LGDRVITTSTRTWALPTYNNHLYKQISSASTGASNDNHYFGYSTPWGYF
DFNRFHCHFSPRDWQRLINNNWGFRPKRLNFKLFNIQVKEVTTNDGVTT
IANNLTSTVQVFSDSEYQLPYVLGSAHQGCLPPFPADVFMIPQYGYLTL
NNGSQAVGRSSFYCLEYFPSQMLRTGNNFTFSYTFEEVPFHSSYAHSQS
LDRLMNPLIDQYLYYLNRTQNQSGSAQNKDLLFSRGSPAGMSVQPKNWL
PGPCYRQQRVSKTKTDNNNSNFTWTGASKYNLNGRESIINPGTAMASHK
DDEDKFFPMSGVMIFGKESAGASNTALDNVMITDEEEIKATNPVATERF
GTVAVNFQSSSTDPATGDVHAMGALPGMVWQDRDVYLQGPIWAKIPHTD
GHFHPSPLMGGFGLKNPPPQILIKNTPVPANPPAEFSATKFASFITQYS
TGQVSVEIEWELQKENSKRWNPEVQYTSNYAKSANVDFTVDNNGLYTEP
RPIGTRYLTRPL

The AAV1 capsid protein VP3 can have the amino acid sequence of SEQ ID NO: 15.

(SEQ ID NO: 15)
MASGGGAPMADNNEGADGVGNASGNWHCDSTWLGDRVITTSTRTWA
LPTYNNHLYKQISSASTGASNDNHYFGYSTPWGYFDFNRFHCHFSPRDW
QRLINNNWGFRPKRLNFKLFNIQVKEVTTNDGVTTIANNLTSTVQVFSD
SEYQLPYVLGSAHQGCLPPFPADVFMIPQYGYLTLNNGSQVGRSSFYCL
EYFPSQMLRTGNNFTFSYTFEEVPFHSSYAHSQSLDRLMNPLIDQYLYY
LNRTQNQSGSAQNKDLLFSRGSPAGMSVQPKNWLPGPCYRQQRVSKTKT
DNNNSNFTWTGASKYNLNGRESIINPGTAMASHKDDEDKFFPMSGVMIF
GKESAGASNTALDNVMITDEEEIKATNPVATERFGTVAVNFQSSSTDPA
TGDVHAMGALPGMVWQDRDVYLQGPIWAKIPHTDGHFHPSPLMGGFGLK
NPPPQILIKNTPVPANPPAEFSATKFASFITQYSTGQVSVEIEWELQKE
NSKRWNPEVQYTSNYAKSANVDFTVDNNGLYTEPRPIGTRYLTRPL

The polynucleotide encoding an AAV1 capsid protein can have the sequence of SEQ ID NO: 10, provided below.

(SEQ ID NO: 10)
ATGGCTGCCGATGGTTATCTTCCAGATTGGCTCGAGGACAACCTCTC
TGAGGGCATTCGCGAGTGGTGGGACTTGAAACCTGGAGCCCCGAAGCCC
AAAGCCAACCAGCAAAAGCAGGACGACGGCCGGGGTCTGGTGCTTCCTG
GCTACAAGTACCTCGGACCCTTCAACGGACTCGACAAGGGGGAGCCCGT
CAACGCGGCGGACGCAGCGGCCCTCGAGCACGACAAGGCCTACGACCAG
CAGCTCAAAGCGGGTGACAATCCGTACCTGCGGTATAACCACGCCGACG
CCGAGTTTCAGGAGCGTCTGCAAGAAGATACGTCTTTTGGGGGCAACCT
CGGGCGAGCAGTCTTCCAGGCCAAGAAGCGGGTTCTCGAACCTCTCGGT
CTGGTTGAGGAAGGCGCTAAGACGGCTCCTGGAAAGAAACGTCCGGTAG
AGCAGTCGCCACAAGAGCCAGACTCCTCCTCGGGCATCGGCAAGACAGG
CCAGCAGCCCGCTAAAAAGAGACTCAATTTTGGTCAGACTGGCGACTCA
GAGTCAGTCCCCGATCCACAACCTCTCGGAGAACCTCCAGCAACCCCCG
CTGCTGTGGGACCTACTACAATGGCTTCAGGCGGTGGCGCACCAATGGC
AGACAATAACGAAGGCGCCGACGGAGTGGGTAATGCCTCAGGAAATTGG
CATTGCGATTCCACATGGCTGGGCGACAGAGTCATCACCACCAGCACCC
GCACCTGGGCCTTGCCCACCTACAATAACCACCTCTACAAGCAAATCTC
CAGTGCTTCAACGGGGGCCAGCAACGACAACCACTACTTCGGCTACAGC
ACCCCCTGGGGGTATTTTGATTTCAACAGATTCCACTGCCACTTTTCAC
CACGTGACTGGCAGCGACTCATCAACAACAATTGGGGATTCCGGCCCAA
GAGACTCAACTTCAAACTCTTCAACATCCAAGTCAAGGAGGTCACGACG
AATGATGGCGTCACAACCATCGCTAATAACCTTACCAGCACGGTTCAAG
TCTTCTCGGACTCGGAGTACCAGCTTCCGTACGTCCTCGGCTCTGCGCA
CCAGGGCTGCCTCCCTCCGTTCCCGGCGGACGTGTTCATGATTCCGCAA
TACGGCTACCTGACGCTCAACAATGGCAGCCAAGCCGTGGGACGTTCAT
CCTTTTACTGCCTGGAATATTTCCCTTCTCAGATGCTGAGAACGGGCAA
CAACTTTACCTTCAGCTACACCTTTGAGGAAGTGCCTTTCCACAGCAGC
TACGCGCACAGCCAGAGCCTGGACCGGCTGATGAATCCTCTCATCGACC
AATACCTGTATTACCTGAACAGAACTCAAAATCAGTCCGGAAGTGCCCA
AAACAAGGACTTGCTGTTTAGCCGTGGGTCTCCAGCTGGCATGTCTGTT
CAGCCCAAAAACTGGCTACCTGGACCCTGTTATCGGCAGCAGCGCGTTT
CTAAAACAAAAACAGACAACAACAACAGCAATTTTACCTGGACTGGTGC
TTCAAAATATAACCTCAATGGGCGTGAATCCATCATCAACCCTGGCACT
GCTATGGCCTCACACAAAGACGACGAAGACAAGTTCTTTCCCATGAGCG
GTGTCATGATTTTTGGAAAAGAGAGCGCCGGAGCTTCAAACACTGCATT
GGACAATGTCATGATTACAGACGAAGAGGAAATTAAAGCCACTAACCCT
GTGGCCACCGAAAGATTTGGGACCGTGGCAGTCAATTTCCAGAGCAGCA
GCACAGACCCTGCGACCGGAGATGTGCATGCTATGGGAGCATTACCTGG
CATGGTGTGGCAAGATAGAGACGTGTACCTGCAGGGTCCCATTTGGGCC
AAAATTCCTCACACAGATGGACACTTTCACCCGTCTCCTCTTATGGGCG
GCTTTGGACTCAAGAACCCGCCTCCTCAGATCCTCATCAAAAACACGCC
TGTTCCTGCGAATCCTCCGGCGGAGTTTTCAGCTACAAAGTTTGCTTCA
TTCATCACCCAATACTCCACAGGACAAGTGAGTGTGGAAATTGAATGGG
AGCTGCAGAAAGAAAACAGCAAGCGCTGGAATCCCGAAGTGCAGTACAC
ATCCAATTATGCAAAATCTGCCAACGTTGATTTTACTGTGGACAACAAT
GGACTTTATACTGAGCCTCGCCCCATTGGCACCCGTTACCTTACCCGTC
CCCTGTAA

The polynucleotide encoding an AAV1 capsid protein can have the sequence of SEQ ID NO: 11, provided below.

(SEQ ID NO: 11)
ATGGCTGCCGATGGTTATCTTCCAGATTGGCTCGAGGACAACCTCTC
TGAGGGCATTCGCGAGTGGTGGGACTTGAAACCTGGAGCCCCGAAGCCC
AAAGCCAACCAGCAAAAGCAGGACGACGGCCGGGGTCTGGTGCTTCCTG
GCTACAAGTACCTCGGACCCTTCAACGGACTCGACAAGGGGGAGCCCGT
CAACGCGGCGGACGCAGCGGCCCTCGAGCACGACAAGGCCTACGACCAG
CAGCTCAAAGCGGGTGACAATCCGTACCTTCGGTATAACCACGCCGACG
CCGAGTTTCAGGAGCGTCTGCAAGAAGATACGTCTTTTGGGGGCAACCT
CGGGCGAGCAGTCTTCCAGGCCAAGAAGCGGGTTCTCGAACCTCTCGGT
CTGGTTGAGGAAGGCGCTAAGACGGCTCCTGGAAAGAAACGTCCGGTAG
AGCAGTCGCCACAAGAGCCAGACTCCTCCTCGGGCATCGGCAAGACAGG
CCAGCAGCCCGCTAAAAAGAGACTCAATTTTGGTCAGACTGGCGACTCA
GAGTCAGTCCCCGACCCACAACCTCTCGGAGAACCTCCAGCAACCCCCG
CTGCTGTGGGACCTACTACAATGGCTTCAGGCGGTGGCGCACCAATGGC
AGACAATAACGAAGGCGCCGACGGAGTGGGTAATGCCTCAGGAAATTGG
CATTGCGATTCCACATGGCTGGGCGACAGAGTCATCACCACCAGCACCC
GCACCTGGGCCTTGCCCACCTACAATAACCACCTCTACAAGCAAATCTC
CAGTGCTTCAACGGGGGCCAGCAACGACAACCACTACTTCGGCTACAGC
ACCCCCTGGGGGTATTTTGATTTCAACAGATTCCACTGCCACTTTTCAC
CACGTGACTGGCAGCGACTCATCAACAACAATTGGGGATTCCGGCCCAA
GAGACTCAACTTCAAACTCTTCAACATCCAAGTCAAGGAGGTCACGACG
AATGATGGCGTCACAACCATCGCTAATAACCTTACCAGCACGGTTCAAG
TCTTCTCGGACTCGGAGTACCAGCTTCCGTACGTCCTCGGCTCTGCGCA
CCAGGGCTGCCTCCCTCCGTTCCCGGCGGACGTGTTCATGATTCCGCAA
TACGGCTACCTGACGCTCAACAATGGCAGCCAAGCCGTGGGACGTTCAT
CCTTTTACTGCCTGGAATATTTCCCTTCTCAGATGCTGAGAACGGGCAA
CAACTTTACCTTCAGCTACACCTTTGAGGAAGTGCCTTTCCACAGCAGC
TACGCGCACAGCCAGAGCCTGGACCGGCTGATGAATCCTCTCATCGACC
AATACCTGTATTACCTGAACAGAACTCAAAATCAGTCCGGAAGTGCCCA
AAACAAGGACTTGCTGTTTAGCCGTGGGTCTCCAGCTGGCATGTCTGTT
CAGCCCAAAAACTGGCTACCTGGACCCTGTTATCGGCAGCAGCGCGTTT
CTAAAACAAAAACAGACAACAACAACAGCAATTTTACCTGGACTGGTGC
TTCAAAATATAACCTCAATGGGCGTGAATCCATCATCAACCCTGGCACT
GCTATGGCCTCACACAAAGACGACGAAGACAAGTTCTTTCCCATGAGCG
GTGTCATGATTTTTGGAAAAGAGAGCGCCGGAGCTTCAAACACTGCATT
GGACAATGTCATGATTACAGACGAAGAGGAAATTAAAGCCACTAACCCT
GTGGCCACCGAAAGATTTGGGACCGTGGCAGTCAATTTCCAGAGCAGCA
GCACAGACCCTGCGACCGGAGATGTGCATGCTATGGGAGCATTACCTGG
CATGGTGTGGCAAGATAGAGACGTGTACCTGCAGGGTCCCATTTGGGCC
AAAATTCCTCACACAGATGGACACTTTCACCCGTCTCCTCTTATGGGCG
GCTTTGGACTCAAGAACCCGCCTCCTCAGATCCTCATCAAAAACACGCC
TGTTCCTGCGAATCCTCCGGCGGAGTTTTCAGCTACAAAGTTTGCTTCA
TTCATCACCCAATACTCCACAGGACAAGTGAGTGTGGAAATTGAATGGG
AGCTGCAGAAAGAAAACAGCAAGCGCTGGAATCCCGAAGTGCAGTACAC
ATCCAATTATGCAAAATCTGCCAACGTTGATTTTACTGTGGACAACAAT
GGACTTTATACTGAGCCTCGCCCCATTGGCACCCGTTACCTTACCCGTC
CCCTG

The modified rep-cap plasmid can contain, in 5′ to 3′ order, a polynucleotide encoding an AAV rep protein (for example, an AAV2 rep protein, such as the polynucleotide of SEQ ID NO: 3), a tetracycline-inducible expression system (for example, a Tet-Off system containing a tTA encoded by SEQ ID NO: 6 or a Tet-On system containing a rtTA encoded by SEQ ID NO: 21 and a series of tetO sequences of SEQ ID NO: 4, such as the series of tetO sequences contained in SEQ ID NO: 7), an AAV promoter (for example, an AAV p41 promoter of SEQ ID NO: 8), and a polynucleotide encoding an AAV1 capsid protein (for example, an AAV1 VP1, VP2, and VP3 protein, such as a polynucleotide of SEQ ID NO: 10 or SEQ ID NO: 11). The modified rep-cap plasmid can include the sequence of SEQ ID NO: 12, provided below.

(SEQ ID NO: 12)
AAACCTGCAGGACTAGAGGTCCTGTATTAGAGGTCACGTGAGTGTTT
TGCGACATTTTGCGACACCATGTGGTCACGCTGGGTATTTAAGCCCGAG
TGAGCACGCAGGGTCTCCATTTTGAAGCGGGAGGTTTGAACGCGCAGCC
GCCAAGCCGAATTCTGCAGATATCCATCACACTGGCGGCCGCTCGACTA
GAGCGGCCGCCACCGCGGTGGAGCTCCAGCTTTTGTTCCCTTTAGTGAG
GGTTAATTGCGCGCTTGGCGTAATCATGGTCATAGCTGTTTCCTGTGTG
AAATTGTTATCCGCTCACAATTCCACACAACATACGAGCCGGAAGCATA
AAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCT
GCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGC
TGCGGCGAGCGGTATCAGCTCACTCAAAGGCGGTAATACGGTTATCCAC
AGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTG
GCGAAACCCGACAGGACTATAAAGATACCAGGCGTTTCCCCCTGGAAGC
TCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATC
GTCTTGAGTCCAACCCGGTAAGACACGACTTATCGCCACTGGCAGCAGC
CACTGGTAACAGGATTAGCAGAGCGAGGTATGTAGGCGGTGCTACAGAG
TTCTTGAAGTGGTGGCCTAACTACGGCTACACTAGAAGAACAGTATTTG
GTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTT
TGCAAGCAGCAGATTACGCGCAGAAAAAAAGGATCTCAAGAAGATCCTT
TGATCTTTTCTACGGGGTCTGACGCTCAGTGGAACGAAAACTCACGTTA
AGGGATTTTGGTCATGAGATTATCAAAAAGGATCTTCACCTAGATCCTT
TTAAATTAAAAATGAAGTTTTAAATCAATCTAAAGTATATATGAGTAAA
CTTGGTCTGACAGTTAGAAAAACTCATCGAGCATCAAATGAAACTGCAA
TTTATTCATATCAGGATTATCAATACCATATTTTTGAAAAAGCCGTTTC
TGTAATGAAGGAGAAAACTCACCGAGGCAGTTCCATAGGATGGCAAGAT
CCTGGTATCGGTCTGCGATTCCGACTCGTCCAACATCAATACAACCTAT
TAATTTCCCCTCGTCAAAAATAAGGTTATCAAGTGAGAAATCACCATGA
GTGACGACTGAATCCGGTGAGAATGGCAAAAGTTTATGCATTTCTTTCC
AGACTTGTTCAACAGGCCAGCCATTACGCTCGTCATCAAAATCACTCGC
ATCAACCAAACCGTTATTCATTCGTGATTGCGCCTGAGCGAGACGAAAT
ACGCGATCGCTGTTAAAAGGACAATTACAAACAGGAATCGAATGCAACC
GGCGCAGGAACACTGCCAGCGCATCAACAATATTTTCACCTGAATCAGG
ATATTCTTCTAATACCTGGAATGCTGTTTTTCCGGGGATCGCAGTGGTG
AGTAACCATGCATCATCAGGAGTACGGATAAAATGCTTGATGGTCGGAA
GAGGCATAAATTCCGTCAGCCAGTTTAGTCTGACCATCTCATCTGTAAC
ATCATTGGCAACGCTACCTTTGCCATGTTTCAGAAACAACTCTGGCGCA
TCGGGCTTCCCATACAAGCGATAGATTGTCGCACCTGATTGCCCGACAT
TATCGCGAGCCCATTTATACCCATATAAATCAGCATCCATGTTGGAATT
TAATCGCGGCCTCGACGTTTCCCGTTGAATATGGCTCATACTCTTCCTT
TTTCAATATTATTGAAGCATTTATCAGGGTTATTGTCTCATGAGCGGAT
ACATATTTGAATGTATTTAGAAAAATAAACAAATAGGGGTTCCGCGCAC
ATTTCCCCGAAAAGTGCCACCTAAATTGTAAGCGTTAATATTTTGTTAA
AATTCGCGTTAAATTTTTGTTAAATCAGCTCATTTTTTAACCAATAGGC
CGAAATCGGCAAAATCCCTTATAAATCAAAAGAATAGACCGAGATAGGG
TTGAGTGTTGTTCCAGTTTGGAACAAGAGTCCACTATTAAAGAACGTGG
ACTCCAACGTCAAAGGGCGAAAAACCGTCTATCAGGGCGATGGCCCACT
ACGTGAACCATCACCCTAATCAAGTTTTTTGGGGTCGAGGTGCCGTAAA
GCACTAAATCGGAACCCTAAAGGGAGCCCCCGATTTAGAGCTTGACGGG
GAAAGCCGGCGAACGTGGCGAGAAAGGAAGGGAAGAAAGCGAAAGGAGC
GGGCGCTAGGGCGCTGGCAAGTGTAGCGGTCACGCTGCGCGTAACCACC
ACACCCGCCGCGCTTAATGCGCCGCTACAGGGCGCGTCCCATTCGCCAT
TCAGGCTGCGCAACTGTTGGGAAGGGCGATCGGTGCGGGCCTCTTCGCT
ATTACGCCAGCTGGCGAAAGGGGGATGTGCTGCAAGGCGATTAAGTTGG
GTAACGCCAGGGTTTTCCCAGTCACGACGTTGTAAAACGACGGCCAGTG
AGCGCGCGTAATACGACTCACTATAGGGCGAATTGGGTACCGGGCCCCC
CCTCGATCGAGGTCGACGGTATCGGGGGAGCTCGCAGGGTCTCCATTTT
GAAGCGGGAGGTTTGAACGCGCAGCCGCCATGCCGGGGTTTTACGAGAT
TGTGATTAAGGTCCCCAGCGACCTTGACGAGCATCTGCCCGGCATTTCT
GACAGCTTTGTGAACTGGGTGGCCGAGAAGGAATGGGAGTTGCCGCCAG
ATTCTGACATGGATCTGAATCTGATTGAGCAGGCACCCCTGACCGTGGC
CGAGAAGCTGCAGCGCGACTTTCTGACGGAATGGCGCCGTGTGAGTAAG
GCCCCGGAGGCTCTTTTCTTTGTGCAATTTGAGAAGGGAGAGAGCTACT
TCCACATGCACGTGCTCGTGGAAACCACCGGGGTGAAATCCATGGTTTT
GGGACGTTTCCTGAGTCAGATTCGCGAAAAACTGATTCAGAGAATTTAC
CGCGGGATCGAGCCGACTTTGCCAAACTGGTTCGCGGTCACAAAGACCA
GAAATGGCGCCGGAGGCGGGAACAAGGTGGTGGATGAGTGCTACATCCC
CAATTACTTGCTCCCCAAAACCCAGCCTGAGCTCCAGTGGGCGTGGACT
AATATGGAACAGTATTTAAGCGCCTGTTTGAATCTCACGGAGCGTAAAC
GGTTGGTGGCGCAGCATCTGACGCACGTGTCGCAGACGCAGGAGCAGAA
CAAAGAGAATCAGAATCCCAATTCTGATGCGCCGGTGATCAGATCAAAA
ACTTCAGCCAGGTACATGGAGCTGGTCGGGTGGCTCGTGGACAAGGGGA
TTACCTCGGAGAAGCAGTGGATCCAGGAGGACCAGGCCTCATACATCTC
CTTCAATGCGGCCTCCAACTCGCGGTCCCAAATCAAGGCTGCCTTGGAC
AATGCGGGAAAGATTATGAGCCTGACTAAAACCGCCCCCGACTACCTGG
TGGGCCAGCAGCCCGTGGAGGACATTTCCAGCAATCGGATTTATAAAAT
TTTGGAACTAAACGGGTACGATCCCCAATATGCGGCTTCCGTCTTTCTG
GGATGGGCCACGAAAAAGTTCGGCAAGAGGAACACCATCTGGCTGTTTG
GGCCTGCAACTACCGGGAAGACCAACATCGCGGAGGCCATAGCCCACAC
TGTGCCCTTCTACGGGTGCGTAAACTGGACCAATGAGAACTTTCCCTTC
AACGACTGTGTCGACAAGATGGTGATCTGGTGGGAGGAGGGGAAGATGA
CCGCCAAGGTCGTGGAGTCGGCCAAAGCCATTCTCGGAGGAAGCAAGGT
GCGCGTGGACCAGAAATGCAAGTCCTCGGCCCAGATAGACCCGACTCCC
GTGATCGTCACCTCCAACACCAACATGTGCGCCGTGATTGACGGGAACT
CAACGACCTTCGAACACCAGCAGCCGTTGCAAGACCGGATGTTCAAATT
TGAACTCACCCGCCGTCTGGATCATGACTTTGGGAAGGTCACCAAGCAG
GAAGTCAAAGACTTTTTCCGGTGGGCAAAGGATCACGTGGTTGAGGTGG
AGCATGAATTCTACGTCAAAAAGGGTGGAGCCAAGAAAAGACCCGCCCC
CAGTGACGCAGATATAAGTGAGCCCAAACGGGTGCGCGAGTCAGTTGCG
CAGCCATCGACGTCAGACGCGGAAGCTTCGATCAACTACGCGGACAGGT
ACCAAAACAAATGTTCTCGTCACGTGGGCATGAATCTGATGCTGTTTCC
CTGCAGACAATGCGAGAGACTGAATCAGAATTCAAATATCTGCTTCACT
CACGGTGTCAAAGACTGTTTAGAGTGCTTTCCCGTGTCAGAATCTCAAC
CCGTTTCTGTCGTCAAAAAGGCGTATCAGAAACTGTGCTACATTCATCA
CATCATGGGAAAGGTGCCAGACGCTTGCACTGCTTGCGACCTGGTCAAT
GTGGACTTGGATGACTGTGTTTCTGAACAATAAATGACTTAAACCAGGT
ATGAGTCGGCTGGATAAATCTAAAGTCATAAACGGCGCTCTGGAATTAC
TCAATGAAGTCGGTATCGAAGGCCTGACGACAAGGAAACTCGCTCAAAA
GCTGGGAGTTGAGCAGCCTACCCTGTACTGGCACGTGAAGAACAAGCGG
GCCCTGCTCGATGCCCTGGCCATCGAGATGCTGGACAGGCATCATACCC
ACTTCTGCCCCCTGGAAGGCGAGTCATGGCAAGACTTTCTGCGGAACAA
CGCCAAGTCATTCCGCTGTGCTCTCCTCTCACATCGCGACGGGGCTAAA
GTGCATCTCGGCACCCGCCCAACAGAGAAACAGTACGAAACCCTGGAAA
ATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAG
GAACAGGAGCATCAAGTAGCAAAAGAGGAAAGAGAGACACCTACCACCG
ATTCTATGCCCCCACTTCTGAGACAAGCAATTGAGCTGTTCGACCGGCA
GGGAGCCGAACCTGCCTTCCTTTTCGGCCTGGAACTAATCATATGTGGC
CTGGAGAAACAGCTAAAGTGCGAAAGCGGCGGGCCGGCCGACGCCCTTG
ACGATTTTGACTTAGACATGCTCCCAGCCGATGCCCTTGACGACTTTGA
CCTTGATATGCTGCCTGCTGACGCTCTTGACGATTTTGACCTTGACATG
CTCCCCGGGTAAATGCATGAATTCGATCTAGAGGGCCCTATTCTATAGT
GTCACCTAAATGCTAGAGCTCGCTGATCAGCCTCGACTGTGCCTTCTAG
TTGCCAGCCATCTGTTGTTTGCCCCTCCCCCGTGCCTTCCTTGACCCTG
GAAGGTGCCACTCCCACTGTCCTTTCCTAATAAAATGAGGAAATTGCAT
CGCATTGTCTGAGTAGGTGTCATTCTATTCTGGGGGGGGGGTGGGGCAG
GACAGCAAGGGGGAGGATTGGGAAGACAATAGCAGGCATGCTGGGGATG
CGGTGGGCTCTATGGCTTCTGAGGCGGAAAGAACCAGCTGGGGCTCGAA
TCAAGCTATCAAGTGCCACCTGACGTCTCCCTATCAGTGATAGAGAAGT
CGACACGTCTCGAGCTCCCTATCAGTGATAGAGAAGGTACGTCTAGAAC
GTCTCCCTATCAGTGATAGAGAAGTCGACACGTCTCGAGCTCCCTATCA
GTGATAGAGAAGGTACGTCTAGAACGTCTCCCTATCAGTGATAGAGAAG
TCGACACGTCTCGAGCTCCCTATCAGTGATAGAGAAGGTACGTCTAGAA
CGTCTCCCTATCAGTGATAGAGAAGTCGACACGTCTCGAGCTCCCTATC
AGTGATAGAGAAGGTACCCCCTATATAAGCAGAGAGATCTGTTCAAATT
TGAACTGACTAAGCGGCTCCCGCCAGATTTTGGCAAGATTACTAAGCAG
GAAGTCAAGGACTTTTTTGCTTGGGCAAAGGTCAATCAGGTGCCGGTGA
CTCACGAGTTTAAAGTTCCCAGGGAATTGGCGGGAACTAAAGGGGCGGA
GAAATCTCTAAAACGCCCACTGGGTGACGTCACCAATACTAGCTATAAA
AGTCTGGAGAAGCGGGCCAGGCTCTCATTTGTTCCCGAGACGCCTCGCA
GTTCAGACGTGACTGTTGATCCCGCTCCTCTGCGACCGCTAGCTTCGAT
CAACTACGCGGACAGGTACCAAAACAAATGTTCTCGTCACGTGGGCATG
AATCTGATGCTGTTTCCCTGCAGACAATGCGAGAGACTGAATCAGAATT
CAAATATCTGCTTCACTCACGGTGTCAAAGACTGTTTAGAGTGCTTTCC
CGTGTCAGAATCTCAACCCGTTTCTGTCGTCAAAAAGGCGTATCAGAAA
CTGTGCTACATTCATCACATCATGGGAAAGGTGCCAGACGCTTGCACTG
CTTGCGACCTGGTCAATGTGGACTTGGATGACTGTGTTTCTGAACAATA
AATGACTTAAACCAGGTATGGCTGCCGATGGTTATCTTCCAGATTGGCT
CGAGGACAACCTCTCTGAGGGCATTCGCGAGTGGTGGGACTTGAAACCT
GGAGCCCCGAAGCCCAAAGCCAACCAGCAAAAGCAGGACGACGGCCGGG
GTCTGGTGCTTCCTGGCTACAAGTACCTCGGACCCTTCAACGGACTCGA
CAAGGGGGAGCCCGTCAACGCGGCGGACGCAGCGGCCCTCGAGCACGAC
AAGGCCTACGACCAGCAGCTCAAAGCGGGTGACAATCCGTACCTGCGGT
ATAACCACGCCGACGCCGAGTTTCAGGAGCGTCTGCAAGAAGATACGTC
TTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAA
AGAAACGTCCGGTAGAGCAGTCGCCACAAGAGCCAGACTCCTCCTCGGG
CATCGGCAAGACAGGCCAGCAGCCCGCTAAAAAGAGACTCAATTTTGGT
CAGACTGGCGACTCAGAGTCAGTCCCCGATCCACAACCTCTCGGAGAAC
CTCCAGCAACCCCCGCTGCTGTGGGACCTACTACAATGGCTTCAGGCGG
TGGCGCACCAATGGCAGACAATAACGAAGGCGCCGACGGAGTGGGTAAT
GCCTCAGGAAATTGGCATTGCGATTCCACATGGCTGGGCGACAGAGTCA
TCACCACCAGCACCCGCACCTGGGCCTTGCCCACCTACAATAACCACCT
CTACAAGCAAATCTCCAGTGCTTCAACGGGGGCCAGCAACGACAACCAC
TACTTCGGCTACAGCACCCCCTGGGGGTATTTTGATTTCAACAGATTCC
ACTGCCACTTTTCACCACGTGACTGGCAGCGACTCATCAACAACAATTG
GGGATTCCGGCCCAAGAGACTCAACTTCAAACTCTTCAACATCCAAGTC
AAGGAGGTCACGACGAATGATGGCGTCACAACCATCGCTAATAACCTTA
CCAGCACGGTTCAAGTCTTCTCGGACTCGGAGTACCAGCTTCCGTACGT
CCTCGGCTCTGCGCACCAGGGCTGCCTCCCTCCGTTCCCGGCGGACGTG
TTCATGATTCCGCAATACGGCTACCTGACGCTCAACAATGGCAGCCAAG
CCGTGGGACGTTCATCCTTTTACTGCCTGGAATATTTCCCTTCTCAGAT
GCTGAGAACGGGCAACAACTTTACCTTCAGCTACACCTTTGAGGAAGTG
CCTTTCCACAGCAGCTACGCGCACAGCCAGAGCCTGGACCGGCTGATGA
ATCCTCTCATCGACCAATACCTGTATTACCTGAACAGAACTCAAAATCA
GTCCGGAAGTGCCCAAAACAAGGACTTGCTGTTTAGCCGTGGGTCTCCA
GCTGGCATGTCTGTTCAGCCCAAAAACTGGCTACCTGGACCCTGTTATC
GGCAGCAGCGCGTTTCTAAAACAAAAACAGACAACAACAACAGCAATTT
TACCTGGACTGGTGCTTCAAAATATAACCTCAATGGGCGTGAATCCATC
ATCAACCCTGGCACTGCTATGGCCTCACACAAAGACGACGAAGACAAGT
TCTTTCCCATGAGCGGTGTCATGATTTTTGGAAAAGAGAGCGCCGGAGC
TTCAAACACTGCATTGGACAATGTCATGATTACAGACGAAGAGGAAATT
AAAGCCACTAACCCTGTGGCCACCGAAAGATTTGGGACCGTGGCAGTCA
ATTTCCAGAGCAGCAGCACAGACCCTGCGACCGGAGATGTGCATGCTAT
GGGAGCATTACCTGGCATGGTGTGGCAAGATAGAGACGTGTACCTGCAG
GGTCCCATTTGGGCCAAAATTCCTCACACAGATGGACACTTTCACCCGT
CTCCTCTTATGGGCGGCTTTGGACTCAAGAACCCGCCTCCTCAGATCCT
CATCAAAAACACGCCTGTTCCTGCGAATCCTCCGGCGGAGTTTTCAGCT
ACAAAGTTTGCTTCATTCATCACCCAATACTCCACAGGACAAGTGAGTG
TGGAAATTGAATGGGAGCTGCAGAAAGAAAACAGCAAGCGCTGGAATCC
CGAAGTGCAGTACACATCCAATTATGCAAAATCTGCCAACGTTGATTTT
ACTGTGGACAACAATGGACTTTATACTGAGCCTCGCCCCATTGGCACCC
GTTACCTTACCCGTCCCCTGTAATAATTGCTTGTTAATCAATAAACCGT
TTAATTCGTTTCAGTTGAACTTTGGTCTCTGCGAAGGGCGAATTCGTTT

The modified rep-cap plasmid can include the sequence of SEQ ID NO: 13, provided below.

(SEQ ID NO: 13)
AAACCTGCAGGACTAGAGGTCCTGTATTAGAGGTCACGTGAGTGTTT
TGCGACATTTTGCGACACCATGTGGTCACGCTGGGTATTTAAGCCCGAG
TGAGCACGCAGGGTCTCCATTTTGAAGCGGGAGGTTTGAACGCGCAGCC
GCCAAGCCGAATTCTGCAGATATCCATCACACTGGCGGCCGCTCGACTA
GAGCGGCCGCCACCGCGGTGGAGCTCCAGCTTTTGTTCCCTTTAGTGAG
GGTTAATTGCGCGCTTGGCGTAATCATGGTCATAGCTGTTTCCTGTGTG
AAATTGTTATCCGCTCACAATTCCACACAACATACGAGCCGGAAGCATA
AAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCT
GCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGC
TGCGGCGAGCGGTATCAGCTCACTCAAAGGCGGTAATACGGTTATCCAC
AGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTG
GCGAAACCCGACAGGACTATAAAGATACCAGGCGTTTCCCCCTGGAAGC
TCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATC
GTCTTGAGTCCAACCCGGTAAGACACGACTTATCGCCACTGGCAGCAGC
CACTGGTAACAGGATTAGCAGAGCGAGGTATGTAGGCGGTGCTACAGAG
TTCTTGAAGTGGTGGCCTAACTACGGCTACACTAGAAGAACAGTATTTG
GTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTT
TGCAAGCAGCAGATTACGCGCAGAAAAAAAGGATCTCAAGAAGATCCTT
TGATCTTTTCTACGGGGTCTGACGCTCAGTGGAACGAAAACTCACGTTA
AGGGATTTTGGTCATGAGATTATCAAAAAGGATCTTCACCTAGATCCTT
TTAAATTAAAAATGAAGTTTTAAATCAATCTAAAGTATATATGAGTAAA
CTTGGTCTGACAGTTAGAAAAACTCATCGAGCATCAAATGAAACTGCAA
TTTATTCATATCAGGATTATCAATACCATATTTTTGAAAAAGCCGTTTC
TGTAATGAAGGAGAAAACTCACCGAGGCAGTTCCATAGGATGGCAAGAT
CCTGGTATCGGTCTGCGATTCCGACTCGTCCAACATCAATACAACCTAT
TAATTTCCCCTCGTCAAAAATAAGGTTATCAAGTGAGAAATCACCATGA
GTGACGACTGAATCCGGTGAGAATGGCAAAAGTTTATGCATTTCTTTCC
AGACTTGTTCAACAGGCCAGCCATTACGCTCGTCATCAAAATCACTCGC
ATCAACCAAACCGTTATTCATTCGTGATTGCGCCTGAGCGAGACGAAAT
ACGCGATCGCTGTTAAAAGGACAATTACAAACAGGAATCGAATGCAACC
GGCGCAGGAACACTGCCAGCGCATCAACAATATTTTCACCTGAATCAGG
ATATTCTTCTAATACCTGGAATGCTGTTTTTCCGGGGATCGCAGTGGTG
AGTAACCATGCATCATCAGGAGTACGGATAAAATGCTTGATGGTCGGAA
GAGGCATAAATTCCGTCAGCCAGTTTAGTCTGACCATCTCATCTGTAAC
ATCATTGGCAACGCTACCTTTGCCATGTTTCAGAAACAACTCTGGCGCA
TCGGGCTTCCCATACAAGCGATAGATTGTCGCACCTGATTGCCCGACAT
TATCGCGAGCCCATTTATACCCATATAAATCAGCATCCATGTTGGAATT
TAATCGCGGCCTCGACGTTTCCCGTTGAATATGGCTCATACTCTTCCTT
TTTCAATATTATTGAAGCATTTATCAGGGTTATTGTCTCATGAGCGGAT
ACATATTTGAATGTATTTAGAAAAATAAACAAATAGGGGTTCCGCGCAC
ATTTCCCCGAAAAGTGCCACCTAAATTGTAAGCGTTAATATTTTGTTAA
AATTCGCGTTAAATTTTTGTTAAATCAGCTCATTTTTTAACCAATAGGC
CGAAATCGGCAAAATCCCTTATAAATCAAAAGAATAGACCGAGATAGGG
TTGAGTGTTGTTCCAGTTTGGAACAAGAGTCCACTATTAAAGAACGTGG
ACTCCAACGTCAAAGGGCGAAAAACCGTCTATCAGGGCGATGGCCCACT
ACGTGAACCATCACCCTAATCAAGTTTTTTGGGGTCGAGGTGCCGTAAA
GCACTAAATCGGAACCCTAAAGGGAGCCCCCGATTTAGAGCTTGACGGG
GAAAGCCGGCGAACGTGGCGAGAAAGGAAGGGAAGAAAGCGAAAGGAGC
GGGCGCTAGGGCGCTGGCAAGTGTAGCGGTCACGCTGCGCGTAACCACC
ACACCCGCCGCGCTTAATGCGCCGCTACAGGGCGCGTCCCATTCGCCAT
TCAGGCTGCGCAACTGTTGGGAAGGGCGATCGGTGCGGGCCTCTTCGCT
ATTACGCCAGCTGGCGAAAGGGGGATGTGCTGCAAGGCGATTAAGTTGG
GTAACGCCAGGGTTTTCCCAGTCACGACGTTGTAAAACGACGGCCAGTG
AGCGCGCGTAATACGACTCACTATAGGGCGAATTGGGTACCGGGCCCCC
CCTCGATCGAGGTCGACGGTATCGGGGGAGCTCGCAGGGTCTCCATTTT
GAAGCGGGAGGTTTGAACGCGCAGCCGCCATGCCGGGGTTTTACGAGAT
TGTGATTAAGGTCCCCAGCGACCTTGACGAGCATCTGCCCGGCATTTCT
GACAGCTTTGTGAACTGGGTGGCCGAGAAGGAATGGGAGTTGCCGCCAG
ATTCTGACATGGATCTGAATCTGATTGAGCAGGCACCCCTGACCGTGGC
CGAGAAGCTGCAGCGCGACTTTCTGACGGAATGGCGCCGTGTGAGTAAG
GCCCCGGAGGCTCTTTTCTTTGTGCAATTTGAGAAGGGAGAGAGCTACT
TCCACATGCACGTGCTCGTGGAAACCACCGGGGTGAAATCCATGGTTTT
GGGACGTTTCCTGAGTCAGATTCGCGAAAAACTGATTCAGAGAATTTAC
CGCGGGATCGAGCCGACTTTGCCAAACTGGTTCGCGGTCACAAAGACCA
GAAATGGCGCCGGAGGGGGGAACAAGGTGGTGGATGAGTGCTACATCCC
CAATTACTTGCTCCCCAAAACCCAGCCTGAGCTCCAGTGGGCGTGGACT
AATATGGAACAGTATTTAAGCGCCTGTTTGAATCTCACGGAGCGTAAAC
GGTTGGTGGCGCAGCATCTGACGCACGTGTCGCAGACGCAGGAGCAGAA
CAAAGAGAATCAGAATCCCAATTCTGATGCGCCGGTGATCAGATCAAAA
ACTTCAGCCAGGTACATGGAGCTGGTCGGGTGGCTCGTGGACAAGGGGA
TTACCTCGGAGAAGCAGTGGATCCAGGAGGACCAGGCCTCATACATCTC
CTTCAATGCGGCCTCCAACTCGCGGTCCCAAATCAAGGCTGCCTTGGAC
AATGCGGGAAAGATTATGAGCCTGACTAAAACCGCCCCCGACTACCTGG
TGGGCCAGCAGCCCGTGGAGGACATTTCCAGCAATCGGATTTATAAAAT
TTTGGAACTAAACGGGTACGATCCCCAATATGCGGCTTCCGTCTTTCTG
GGATGGGCCACGAAAAAGTTCGGCAAGAGGAACACCATCTGGCTGTTTG
GGCCTGCAACTACCGGGAAGACCAACATCGCGGAGGCCATAGCCCACAC
TGTGCCCTTCTACGGGTGCGTAAACTGGACCAATGAGAACTTTCCCTTC
AACGACTGTGTCGACAAGATGGTGATCTGGTGGGAGGAGGGGAAGATGA
CCGCCAAGGTCGTGGAGTCGGCCAAAGCCATTCTCGGAGGAAGCAAGGT
GCGCGTGGACCAGAAATGCAAGTCCTCGGCCCAGATAGACCCGACTCCC
GTGATCGTCACCTCCAACACCAACATGTGCGCCGTGATTGACGGGAACT
CAACGACCTTCGAACACCAGCAGCCGTTGCAAGACCGGATGTTCAAATT
TGAACTCACCCGCCGTCTGGATCATGACTTTGGGAAGGTCACCAAGCAG
GAAGTCAAAGACTTTTTCCGGTGGGCAAAGGATCACGTGGTTGAGGTGG
AGCATGAATTCTACGTCAAAAAGGGTGGAGCCAAGAAAAGACCCGCCCC
CAGTGACGCAGATATAAGTGAGCCCAAACGGGTGCGCGAGTCAGTTGCG
CAGCCATCGACGTCAGACGCGGAAGCTTCGATCAACTACGCGGACAGGT
ACCAAAACAAATGTTCTCGTCACGTGGGCATGAATCTGATGCTGTTTCC
CTGCAGACAATGCGAGAGACTGAATCAGAATTCAAATATCTGCTTCACT
CACGGTGTCAAAGACTGTTTAGAGTGCTTTCCCGTGTCAGAATCTCAAC
CCGTTTCTGTCGTCAAAAAGGCGTATCAGAAACTGTGCTACATTCATCA
CATCATGGGAAAGGTGCCAGACGCTTGCACTGCTTGCGACCTGGTCAAT
GTGGACTTGGATGACTGTGTTTCTGAACAATAAATGACTTAAACCAGGT
ATGAGTCGGCTGGATAAATCTAAAGTCATAAACGGCGCTCTGGAATTAC
TCAATGAAGTCGGTATCGAAGGCCTGACGACAAGGAAACTCGCTCAAAA
GCTGGGAGTTGAGCAGCCTACCCTGTACTGGCACGTGAAGAACAAGCGG
GCCCTGCTCGATGCCCTGGCCATCGAGATGCTGGACAGGCATCATACCC
ACTTCTGCCCCCTGGAAGGCGAGTCATGGCAAGACTTTCTGCGGAACAA
CGCCAAGTCATTCCGCTGTGCTCTCCTCTCACATCGCGACGGGGCTAAA
GTGCATCTCGGCACCCGCCCAACAGAGAAACAGTACGAAACCCTGGAAA
ATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAG
GAACAGGAGCATCAAGTAGCAAAAGAGGAAAGAGAGACACCTACCACCG
ATTCTATGCCCCCACTTCTGAGACAAGCAATTGAGCTGTTCGACCGGCA
GGGAGCCGAACCTGCCTTCCTTTTCGGCCTGGAACTAATCATATGTGGC
CTGGAGAAACAGCTAAAGTGCGAAAGCGGCGGGCCGGCCGACGCCCTTG
ACGATTTTGACTTAGACATGCTCCCAGCCGATGCCCTTGACGACTTTGA
CCTTGATATGCTGCCTGCTGACGCTCTTGACGATTTTGACCTTGACATG
CTCCCCGGGTAAATGCATGAATTCGATCTAGAGGGCCCTATTCTATAGT
GTCACCTAAATGCTAGAGCTCGCTGATCAGCCTCGACTGTGCCTTCTAG
TTGCCAGCCATCTGTTGTTTGCCCCTCCCCCGTGCCTTCCTTGACCCTG
GAAGGTGCCACTCCCACTGTCCTTTCCTAATAAAATGAGGAAATTGCAT
CGCATTGTCTGAGTAGGTGTCATTCTATTCTGGGGGGGGGGGGGGCAGG
ACAGCAAGGGGGAGGATTGGGAAGACAATAGCAGGCATGCTGGGGATGC
GGTGGGCTCTATGGCTTCTGAGGCGGAAAGAACCAGCTGGGGCTCGAAT
CAAGCTATCAAGTGCCACCTGACGTCTCCCTATCAGTGATAGAGAAGTC
GACACGTCTCGAGCTCCCTATCAGTGATAGAGAAGGTACGTCTAGAACG
TCTCCCTATCAGTGATAGAGAAGTCGACACGTCTCGAGCTCCCTATCAG
TGATAGAGAAGGTACGTCTAGAACGTCTCCCTATCAGTGATAGAGAAGT
CGACACGTCTCGAGCTCCCTATCAGTGATAGAGAAGGTACGTCTAGAAC
GTCTCCCTATCAGTGATAGAGAAGTCGACACGTCTCGAGCTCCCTATCA
GTGATAGAGAAGGTACCCCCTATATAAGCAGAGAGATCTGTTCAAATTT
GAACTGACTAAGCGGCTCCCGCCAGATTTTGGCAAGATTACTAAGCAGG
AAGTCAAGGACTTTTTTGCTTGGGCAAAGGTCAATCAGGTGCCGGTGAC
TCACGAGTTTAAAGTTCCCAGGGAATTGGCGGGAACTAAAGGGGCGGAG
AAATCTCTAAAACGCCCACTGGGTGACGTCACCAATACTAGCTATAAAA
GTCTGGAGAAGCGGGCCAGGCTCTCATTTGTTCCCGAGACGCCTCGCAG
TTCAGACGTGACTGTTGATCCCGCTCCTCTGCGACCGCTAGCTTCGATC
AACTACGCGGACAGGTACCAAAACAAATGTTCTCGTCACGTGGGCATGA
ATCTGATGCTGTTTCCCTGCAGACAATGCGAGAGACTGAATCAGAATTC
AAATATCTGCTTCACTCACGGTGTCAAAGACTGTTTAGAGTGCTTTCCC
GTGTCAGAATCTCAACCCGTTTCTGTCGTCAAAAAGGCGTATCAGAAAC
TGTGCTACATTCATCACATCATGGGAAAGGTGCCAGACGCTTGCACTGC
TTGCGACCTGGTCAATGTGGACTTGGATGACTGTGTTTCTGAACAATAA
ATGACTTAAACCAGGTATGGCTGCCGATGGTTATCTTCCAGATTGGCTC
GAGGACAACCTCTCTGAGGGCATTCGCGAGTGGTGGGACTTGAAACCTG
GAGCCCCGAAGCCCAAAGCCAACCAGCAAAAGCAGGACGACGGCCGGGG
TCTGGTGCTTCCTGGCTACAAGTACCTCGGACCCTTCAACGGACTCGAC
AAGGGGGAGCCCGTCAACGCGGCGGACGCAGCGGCCCTCGAGCACGACA
AGGCCTACGACCAGCAGCTCAAAGCGGGTGACAATCCGTACCTTCGGTA
TAACCACGCCGACGCCGAGTTTCAGGAGCGTCTGCAAGAAGATACGTCT
TTTGGGGGCAACCTCGGGCGAGCAGTCTTCCAGGCCAAGAAGCGGGTTC
TCGAACCTCTCGGTCTGGTTGAGGAAGGCGCTAAGACGGCTCCTGGAAA
GAAACGTCCGGTAGAGCAGTCGCCACAAGAGCCAGACTCCTCCTCGGGC
ATCGGCAAGACAGGCCAGCAGCCCGCTAAAAAGAGACTCAATTTTGGTC
AGACTGGCGACTCAGAGTCAGTCCCCGACCCACAACCTCTCGGAGAACC
TCCAGCAACCCCCGCTGCTGTGGGACCTACTACAATGGCTTCAGGCGGT
GGCGCACCAATGGCAGACAATAACGAAGGCGCCGACGGAGTGGGTAATG
CCTCAGGAAATTGGCATTGCGATTCCACATGGCTGGGCGACAGAGTCAT
CACCACCAGCACCCGCACCTGGGCCTTGCCCACCTACAATAACCACCTC
TACAAGCAAATCTCCAGTGCTTCAACGGGGGCCAGCAACGACAACCACT
ACTTCGGCTACAGCACCCCCTGGGGGTATTTTGATTTCAACAGATTCCA
CTGCCACTTTTCACCACGTGACTGGCAGCGACTCATCAACAACAATTGG
GGATTCCGGCCCAAGAGACTCAACTTCAAACTCTTCAACATCCAAGTCA
AGGAGGTCACGACGAATGATGGCGTCACAACCATCGCTAATAACCTTAC
CAGCACGGTTCAAGTCTTCTCGGACTCGGAGTACCAGCTTCCGTACGTC
CTCGGCTCTGCGCACCAGGGCTGCCTCCCTCCGTTCCCGGCGGACGTGT
TCATGATTCCGCAATACGGCTACCTGACGCTCAACAATGGCAGCCAAGC
CGTGGGACGTTCATCCTTTTACTGCCTGGAATATTTCCCTTCTCAGATG
CTGAGAACGGGCAACAACTTTACCTTCAGCTACACCTTTGAGGAAGTGC
CTTTCCACAGCAGCTACGCGCACAGCCAGAGCCTGGACCGGCTGATGAA
TCCTCTCATCGACCAATACCTGTATTACCTGAACAGAACTCAAAATCAG
TCCGGAAGTGCCCAAAACAAGGACTTGCTGTTTAGCCGTGGGTCTCCAG
CTGGCATGTCTGTTCAGCCCAAAAACTGGCTACCTGGACCCTGTTATCG
GCAGCAGCGCGTTTCTAAAACAAAAACAGACAACAACAACAGCAATTTT
ACCTGGACTGGTGCTTCAAAATATAACCTCAATGGGCGTGAATCCATCA
TCAACCCTGGCACTGCTATGGCCTCACACAAAGACGACGAAGACAAGTT
CTTTCCCATGAGCGGTGTCATGATTTTTGGAAAAGAGAGCGCCGGAGCT
TCAAACACTGCATTGGACAATGTCATGATTACAGACGAAGAGGAAATTA
AAGCCACTAACCCTGTGGCCACCGAAAGATTTGGGACCGTGGCAGTCAA
TTTCCAGAGCAGCAGCACAGACCCTGCGACCGGAGATGTGCATGCTATG
GGAGCATTACCTGGCATGGTGTGGCAAGATAGAGACGTGTACCTGCAGG
GTCCCATTTGGGCCAAAATTCCTCACACAGATGGACACTTTCACCCGTC
TCCTCTTATGGGCGGCTTTGGACTCAAGAACCCGCCTCCTCAGATCCTC
ATCAAAAACACGCCTGTTCCTGCGAATCCTCCGGCGGAGTTTTCAGCTA
CAAAGTTTGCTTCATTCATCACCCAATACTCCACAGGACAAGTGAGTGT
GGAAATTGAATGGGAGCTGCAGAAAGAAAACAGCAAGCGCTGGAATCCC
GAAGTGCAGTACACATCCAATTATGCAAAATCTGCCAACGTTGATTTTA
CTGTGGACAACAATGGACTTTATACTGAGCCTCGCCCCATTGGCACCCG
TTACCTTACCCGTCCCCTGTAATTGCTTGTTAATCAATAAACCGTTTAA
TTCGTTTCAGTTGAACTTTGGTCTCTGCGAAGGGCGAATTCGTTT

The modified rep-cap plasmid can include the sequence of SEQ ID NO: 22, provided below.

(SEQ ID NO: 22)
AAACCTGCAGGACTAGAGGTCCTGTATTAGAGGTCACGTGAGTGTTT
TGCGACATTTTGCGACACCATGTGGTCACGCTGGGTATTTAAGCCCGAG
TGAGCACGCAGGGTCTCCATTTTGAAGCGGGAGGTTTGAACGCGCAGCC
GCCAAGCCGAATTCTGCAGATATCCATCACACTGGCGGCCGCTCGACTA
GAGCGGCCGCCACCGCGGTGGAGCTCCAGCTTTTGTTCCCTTTAGTGAG
GGTTAATTGCGCGCTTGGCGTAATCATGGTCATAGCTGTTTCCTGTGTG
AAATTGTTATCCGCTCACAATTCCACACAACATACGAGCCGGAAGCATA
AAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCT
GCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGC
TGCGGCGAGCGGTATCAGCTCACTCAAAGGCGGTAATACGGTTATCCAC
AGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTG
GCGAAACCCGACAGGACTATAAAGATACCAGGCGTTTCCCCCTGGAAGC
TCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATC
GTCTTGAGTCCAACCCGGTAAGACACGACTTATCGCCACTGGCAGCAGC
CACTGGTAACAGGATTAGCAGAGCGAGGTATGTAGGCGGTGCTACAGAG
TTCTTGAAGTGGTGGCCTAACTACGGCTACACTAGAAGAACAGTATTTG
GTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTT
TGCAAGCAGCAGATTACGCGCAGAAAAAAAGGATCTCAAGAAGATCCTT
TGATCTTTTCTACGGGGTCTGACGCTCAGTGGAACGAAAACTCACGTTA
AGGGATTTTGGTCATGAGATTATCAAAAAGGATCTTCACCTAGATCCTT
TTAAATTAAAAATGAAGTTTTAAATCAATCTAAAGTATATATGAGTAAA
CTTGGTCTGACAGTTAGAAAAACTCATCGAGCATCAAATGAAACTGCAA
TTTATTCATATCAGGATTATCAATACCATATTTTTGAAAAAGCCGTTTC
TGTAATGAAGGAGAAAACTCACCGAGGCAGTTCCATAGGATGGCAAGAT
CCTGGTATCGGTCTGCGATTCCGACTCGTCCAACATCAATACAACCTAT
TAATTTCCCCTCGTCAAAAATAAGGTTATCAAGTGAGAAATCACCATGA
GTGACGACTGAATCCGGTGAGAATGGCAAAAGTTTATGCATTTCTTTCC
AGACTTGTTCAACAGGCCAGCCATTACGCTCGTCATCAAAATCACTCGC
ATCAACCAAACCGTTATTCATTCGTGATTGCGCCTGAGCGAGACGAAAT
ACGCGATCGCTGTTAAAAGGACAATTACAAACAGGAATCGAATGCAACC
GGCGCAGGAACACTGCCAGCGCATCAACAATATTTTCACCTGAATCAGG
ATATTCTTCTAATACCTGGAATGCTGTTTTTCCGGGGATCGCAGTGGTG
AGTAACCATGCATCATCAGGAGTACGGATAAAATGCTTGATGGTCGGAA
GAGGCATAAATTCCGTCAGCCAGTTTAGTCTGACCATCTCATCTGTAAC
ATCATTGGCAACGCTACCTTTGCCATGTTTCAGAAACAACTCTGGCGCA
TCGGGCTTCCCATACAAGCGATAGATTGTCGCACCTGATTGCCCGACAT
TATCGCGAGCCCATTTATACCCATATAAATCAGCATCCATGTTGGAATT
TAATCGCGGCCTCGACGTTTCCCGTTGAATATGGCTCATACTCTTCCTT
TTTCAATATTATTGAAGCATTTATCAGGGTTATTGTCTCATGAGCGGAT
ACATATTTGAATGTATTTAGAAAAATAAACAAATAGGGGTTCCGCGCAC
ATTTCCCCGAAAAGTGCCACCTAAATTGTAAGCGTTAATATTTTGTTAA
AATTCGCGTTAAATTTTTGTTAAATCAGCTCATTTTTTAACCAATAGGC
CGAAATCGGCAAAATCCCTTATAAATCAAAAGAATAGACCGAGATAGGG
TTGAGTGTTGTTCCAGTTTGGAACAAGAGTCCACTATTAAAGAACGTGG
ACTCCAACGTCAAAGGGCGAAAAACCGTCTATCAGGGCGATGGCCCACT
ACGTGAACCATCACCCTAATCAAGTTTTTTGGGGTCGAGGTGCCGTAAA
GCACTAAATCGGAACCCTAAAGGGAGCCCCCGATTTAGAGCTTGACGGG
GAAAGCCGGCGAACGTGGCGAGAAAGGAAGGGAAGAAAGCGAAAGGAGC
GGGCGCTAGGGCGCTGGCAAGTGTAGCGGTCACGCTGCGCGTAACCACC
ACACCCGCCGCGCTTAATGCGCCGCTACAGGGCGCGTCCCATTCGCCAT
TCAGGCTGCGCAACTGTTGGGAAGGGCGATCGGTGCGGGCCTCTTCGCT
ATTACGCCAGCTGGCGAAAGGGGGATGTGCTGCAAGGCGATTAAGTTGG
GTAACGCCAGGGTTTTCCCAGTCACGACGTTGTAAAACGACGGCCAGTG
AGCGCGCGTAATACGACTCACTATAGGGCGAATTGGGTACCGGGCCCCC
CCTCGATCGAGGTCGACGGTATCGGGGGAGCTCGCAGGGTCTCCATTTT
GAAGCGGGAGGTTTGAACGCGCAGCCGCCATGCCGGGGTTTTACGAGAT
TGTGATTAAGGTCCCCAGCGACCTTGACGAGCATCTGCCCGGCATTTCT
GACAGCTTTGTGAACTGGGTGGCCGAGAAGGAATGGGAGTTGCCGCCAG
ATTCTGACATGGATCTGAATCTGATTGAGCAGGCACCCCTGACCGTGGC
CGAGAAGCTGCAGCGCGACTTTCTGACGGAATGGCGCCGTGTGAGTAAG
GCCCCGGAGGCTCTTTTCTTTGTGCAATTTGAGAAGGGAGAGAGCTACT
TCCACATGCACGTGCTCGTGGAAACCACCGGGGTGAAATCCATGGTTTT
GGGACGTTTCCTGAGTCAGATTCGCGAAAAACTGATTCAGAGAATTTAC
CGCGGGATCGAGCCGACTTTGCCAAACTGGTTCGCGGTCACAAAGACCA
GAAATGGCGCCGGAGGCGGGAACAAGGTGGTGGATGAGTGCTACATCCC
CAATTACTTGCTCCCCAAAACCCAGCCTGAGCTCCAGTGGGCGTGGACT
AATATGGAACAGTATTTAAGCGCCTGTTTGAATCTCACGGAGCGTAAAC
GGTTGGTGGCGCAGCATCTGACGCACGTGTCGCAGACGCAGGAGCAGAA
CAAAGAGAATCAGAATCCCAATTCTGATGCGCCGGTGATCAGATCAAAA
ACTTCAGCCAGGTACATGGAGCTGGTCGGGTGGCTCGTGGACAAGGGGA
TTACCTCGGAGAAGCAGTGGATCCAGGAGGACCAGGCCTCATACATCTC
CTTCAATGCGGCCTCCAACTCGCGGTCCCAAATCAAGGCTGCCTTGGAC
AATGCGGGAAAGATTATGAGCCTGACTAAAACCGCCCCCGACTACCTGG
TGGGCCAGCAGCCCGTGGAGGACATTTCCAGCAATCGGATTTATAAAAT
TTTGGAACTAAACGGGTACGATCCCCAATATGCGGCTTCCGTCTTTCTG
GGATGGGCCACGAAAAAGTTCGGCAAGAGGAACACCATCTGGCTGTTTG
GGCCTGCAACTACCGGGAAGACCAACATCGCGGAGGCCATAGCCCACAC
TGTGCCCTTCTACGGGTGCGTAAACTGGACCAATGAGAACTTTCCCTTC
AACGACTGTGTCGACAAGATGGTGATCTGGTGGGAGGAGGGGAAGATGA
CCGCCAAGGTCGTGGAGTCGGCCAAAGCCATTCTCGGAGGAAGCAAGGT
GCGCGTGGACCAGAAATGCAAGTCCTCGGCCCAGATAGACCCGACTCCC
GTGATCGTCACCTCCAACACCAACATGTGCGCCGTGATTGACGGGAACT
CAACGACCTTCGAACACCAGCAGCCGTTGCAAGACCGGATGTTCAAATT
TGAACTCACCCGCCGTCTGGATCATGACTTTGGGAAGGTCACCAAGCAG
GAAGTCAAAGACTTTTTCCGGTGGGCAAAGGATCACGTGGTTGAGGTGG
AGCATGAATTCTACGTCAAAAAGGGTGGAGCCAAGAAAAGACCCGCCCC
CAGTGACGCAGATATAAGTGAGCCCAAACGGGTGCGCGAGTCAGTTGCG
CAGCCATCGACGTCAGACGCGGAAGCTTCGATCAACTACGCGGACAGGT
ACCAAAACAAATGTTCTCGTCACGTGGGCATGAATCTGATGCTGTTTCC
CTGCAGACAATGCGAGAGACTGAATCAGAATTCAAATATCTGCTTCACT
CACGGTGTCAAAGACTGTTTAGAGTGCTTTCCCGTGTCAGAATCTCAAC
CCGTTTCTGTCGTCAAAAAGGCGTATCAGAAACTGTGCTACATTCATCA
CATCATGGGAAAGGTGCCAGACGCTTGCACTGCTTGCGACCTGGTCAAT
GTGGACTTGGATGACTGTGTTTCTGAACAATAAATGACTTAAACCAGGT
ATGAGTCGGCTGGATAAATCTAAAGTCATAAACTCTGCTCTGGAATTAC
TCAATGGAGTCGGTATCGAAGGCCTGACGACAAGGAAACTCGCTCAAAA
GCTGGGAGTTGAGCAGCCTACCCTGTACTGGCACGTGAAGAACAAGCGG
GCCCTGCTCGATGCCCTGCCAATCGAGATGCTGGACAGGCATCATACCC
ACTCCTGCCCCCTGGAAGGCGAGTCATGGCAAGACTTTCTGCGGAACAA
CGCCAAGTCATACCGCTGTGCTCTCCTCTCACATCGCGACGGGGCTAAA
GTGCATCTCGGCACCCGCCCAACAGAGAAACAGTACGAAACCCTGGAAA
ATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAG
GAACAGGAGCATCAAGTAGCAAAAGAGGAAAGAGAGACACCTACCACCG
ATTCTATGCCCCCACTTCTGAAACAAGCAATTGAGCTGTTCGACCGGCA
GCTCCCAGCCGATGCCCTTGACGACTTTGACCTTGATATGCTGCCTGCT
GGGGAGCCGAACCTGCCTTCCTTTTCGGCCTGGAACTAATCATATGTGG
CCTGGAGAAACAGCTAAAGTGCGAAAGCGGCGGGCCGACCGACGCCCTT
GACGATTTTGACTTAGACATACGCTCTTGACGATTTTGACCTTGACATG
CTCCCCGGGTAAATGCATGAATTCGATCTAGAGGGCCCTATTCTATAGT
GTCACCTAAATGCTAGAGCTCGCTGATCAGCCTCGACTGTGCCTTCTAG
TTGCCAGCCATCTGTTGTTTGCCCCTCCCCCGTGCCTTCCTTGACCCTG
GAAGGTGCCACTCCCACTGTCCTTTCCTAATAAAATGAGGAAATTGCAT
CGCATTGTCTGAGTAGGTGTCATTCTATTCTGGGGGGGGGGTGGGGCAG
GACAGCAAGGGGGAGGATTGGGAAGACAATAGCAGGCATGCTGGGGATG
CGGTGGGCTCTATGGCTTCTGAGGCGGAAAGAACCAGCTGGGGCTCGAA
TCAAGCTATCAAGTGCCACCTGACGTCTCCCTATCAGTGATAGAGAAGT
CGACACGTCTCGAGCTCCCTATCAGTGATAGAGAAGGTACGTCTAGAAC
GTCTCCCTATCAGTGATAGAGAAGTCGACACGTCTCGAGCTCCCTATCA
GTGATAGAGAAGGTACGTCTAGAACGTCTCCCTATCAGTGATAGAGAAG
TCGACACGTCTCGAGCTCCCTATCAGTGATAGAGAAGGTACGTCTAGAA
CGTCTCCCTATCAGTGATAGAGAAGTCGACACGTCTCGAGCTCCCTATC
AGTGATAGAGAAGGTACCCCCTATATAAGCAGAGAGATCTGTTCAAATT
TGAACTGACTAAGCGGCTCCCGCCAGATTTTGGCAAGATTACTAAGCAG
GAAGTCAAGGACTTTTTTGCTTGGGCAAAGGTCAATCAGGTGCCGGTGA
CTCACGAGTTTAAAGTTCCCAGGGAATTGGCGGGAACTAAAGGGGCGGA
GAAATCTCTAAAACGCCCACTGGGTGACGTCACCAATACTAGCTATAAA
AGTCTGGAGAAGCGGGCCAGGCTCTCATTTGTTCCCGAGACGCCTCGCA
GTTCAGACGTGACTGTTGATCCCGCTCCTCTGCGACCGCTAGCTTCGAT
CAACTACGCGGACAGGTACCAAAACAAATGTTCTCGTCACGTGGGCATG
AATCTGATGCTGTTTCCCTGCAGACAATGCGAGAGACTGAATCAGAATT
CAAATATCTGCTTCACTCACGGTGTCAAAGACTGTTTAGAGTGCTTTCC
CGTGTCAGAATCTCAACCCGTTTCTGTCGTCAAAAAGGCGTATCAGAAA
CTGTGCTACATTCATCACATCATGGGAAAGGTGCCAGACGCTTGCACTG
CTTGCGACCTGGTCAATGTGGACTTGGATGACTGTGTTTCTGAACAATA
AATGACTTAAACCAGGTATGGCTGCCGATGGTTATCTTCCAGATTGGCT
CGAGGACAACCTCTCTGAGGGCATTCGCGAGTGGTGGGACTTGAAACCT
GGAGCCCCGAAGCCCAAAGCCAACCAGCAAAAGCAGGACGACGGCCGGG
GTCTGGTGCTTCCTGGCTACAAGTACCTCGGACCCTTCAACGGACTCGA
CAAGGGGGAGCCCGTCAACGCGGCGGACGCAGCGGCCCTCGAGCACGAC
AAGGCCTACGACCAGCAGCTCAAAGCGGGTGACAATCCGTACCTGCGGT
ATAACCACGCCGACGCCGAGTTTCAGGAGCGTCTGCAAGAAGATACGTC
TTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAA
AGAAACGTCCGGTAGAGCAGTCGCCACAAGAGCCAGACTCCTCCTCGGG
CATCGGCAAGACAGGCCAGCAGCCCGCTAAAAAGAGACTCAATTTTGGT
CAGACTGGCGACTCAGAGTCAGTCCCCGATCCACAACCTCTCGGAGAAC
CTCCAGCAACCCCCGCTGCTGTGGGACCTACTACAATGGCTTCAGGCGG
TGGCGCACCAATGGCAGACAATAACGAAGGCGCCGACGGAGTGGGTAAT
GCCTCAGGAAATTGGCATTGCGATTCCACATGGCTGGGCGACAGAGTCA
TCACCACCAGCACCCGCACCTGGGCCTTGCCCACCTACAATAACCACCT
CTACAAGCAAATCTCCAGTGCTTCAACGGGGGCCAGCAACGACAACCAC
TACTTCGGCTACAGCACCCCCTGGGGGTATTTTGATTTCAACAGATTCC
ACTGCCACTTTTCACCACGTGACTGGCAGCGACTCATCAACAACAATTG
GGGATTCCGGCCCAAGAGACTCAACTTCAAACTCTTCAACATCCAAGTC
AAGGAGGTCACGACGAATGATGGCGTCACAACCATCGCTAATAACCTTA
CCAGCACGGTTCAAGTCTTCTCGGACTCGGAGTACCAGCTTCCGTACGT
CCTCGGCTCTGCGCACCAGGGCTGCCTCCCTCCGTTCCCGGCGGACGTG
TTCATGATTCCGCAATACGGCTACCTGACGCTCAACAATGGCAGCCAAG
CCGTGGGACGTTCATCCTTTTACTGCCTGGAATATTTCCCTTCTCAGAT
GCTGAGAACGGGCAACAACTTTACCTTCAGCTACACCTTTGAGGAAGTG
CCTTTCCACAGCAGCTACGCGCACAGCCAGAGCCTGGACCGGCTGATGA
ATCCTCTCATCGACCAATACCTGTATTACCTGAACAGAACTCAAAATCA
GTCCGGAAGTGCCCAAAACAAGGACTTGCTGTTTAGCCGTGGGTCTCCA
GCTGGCATGTCTGTTCAGCCCAAAAACTGGCTACCTGGACCCTGTTATC
GGCAGCAGCGCGTTTCTAAAACAAAAACAGACAACAACAACAGCAATTT
TACCTGGACTGGTGCTTCAAAATATAACCTCAATGGGCGTGAATCCATC
ATCAACCCTGGCACTGCTATGGCCTCACACAAAGACGACGAAGACAAGT
TCTTTCCCATGAGCGGTGTCATGATTTTTGGAAAAGAGAGCGCCGGAGC
TTCAAACACTGCATTGGACAATGTCATGATTACAGACGAAGAGGAAATT
AAAGCCACTAACCCTGTGGCCACCGAAAGATTTGGGACCGTGGCAGTCA
ATTTCCAGAGCAGCAGCACAGACCCTGCGACCGGAGATGTGCATGCTAT
GGGAGCATTACCTGGCATGGTGTGGCAAGATAGAGACGTGTACCTGCAG
GGTCCCATTTGGGCCAAAATTCCTCACACAGATGGACACTTTCACCCGT
CTCCTCTTATGGGCGGCTTTGGACTCAAGAACCCGCCTCCTCAGATCCT
CATCAAAAACACGCCTGTTCCTGCGAATCCTCCGGCGGAGTTTTCAGCT
ACAAAGTTTGCTTCATTCATCACCCAATACTCCACAGGACAAGTGAGTG
TGGAAATTGAATGGGAGCTGCAGAAAGAAAACAGCAAGCGCTGGAATCC
CGAAGTGCAGTACACATCCAATTATGCAAAATCTGCCAACGTTGATTTT
ACTGTGGACAACAATGGACTTTATACTGAGCCTCGCCCCATTGGCACCC
GTTACCTTACCCGTCCCCTGTAATAATTGCTTGTTAATCAATAAACCGT
TTAATTCGTTTCAGTTGAACTTTGGTCTCTGCGAAGGGCGAATTCGTT
T.

### Methods for the Delivery of Exogenous Polynucleotides to Target Host Cells

The modified rep-cap plasmids described herein can be introduced into a cell along with a helper plasmid and a transgene plasmid to produce an rAAV vector (for example, rAAV1 vector) using methods known in the art. Techniques that can be used to introduce a polynucleotide, such as DNA and RNA (for example, encoding a rep protein, tetracycline inducible expression system, and AAV1 capsid protein described herein), into a target host cell (for example, a mammalian cell) are well known in the art. For example, electroporation can be used to permeabilize mammalian cells by the application of an electrostatic potential to the cell of interest. Mammalian cells, such as human cells, subjected to an external electric field in this manner are subsequently predisposed to the uptake of exogenous nucleic acids (for example, nucleic acids capable of expression in cells). Electroporation of mammalian cells is described in detail, for example, in Chu et al., Nucleic Acids Research 15:1311 (1987). A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, for example, in Distler et al., Experimental Dermatology 14:315 (2005), as well as in US 2010/0317114.

An additional technique useful for the transfection of target host cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target host cell. Squeeze-poration is described in detail, for example, in Sharei et al., Journal of Visualized Experiments 81: e50980 (2013).

Lipofection represents another technique useful for transfection of target host cells. This method involves the loading of nucleic acids into a liposome, which often presents cationic functional groups, such as quaternary and protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane and by endocytosis of the complex. Lipofection is described in detail, for example, in U.S. Pat. No. 7,442,386. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, for example, in Dennig, Topics in Current Chemistry 228:227 (2003)) polyethylenimine, and DEAE-dextran, the use of which as a transfection agent is described in detail, for example, in Gulick et al., Current Protocols in Molecular Biology 40:1:9.2:9.2.1 (1997).

Another useful tool for inducing the uptake of exogenous nucleic acids by target host cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target host cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells and tissue. Cells that are impaled by nanostructures can express the delivered gene(s). An example of this technique is described in Shalek et al., PNAS 107:25 1870 (2010).

MAGNETOFECTION™ can also be used to deliver nucleic acids to target host cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (for example, a plasmid or viral vector) is achieved by salt-induced colloidal aggregation and electrostatic interaction. The magnetic particles are then concentrated on the target host cells by the influence of an external magnetic field generated by magnets. This technique is described in detail in Scherer et al., Gene Therapy 9:102 (2002). Magnetic beads are another tool that can be used to transfect target host cells in a mild and efficient manner, as this methodology utilizes an applied magnetic field in order to direct the uptake of nucleic acids. This technology is described in detail, for example, in US2010/0227406.

Another useful tool for inducing the uptake of exogenous nucleic acids by target host cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, for example, in Rhodes et al., Methods in Cell Biology 82:309 (2007).

Microvesicles represent another potential vehicle that can be used to modify the genome of a target host cell according to the methods described herein. For example, microvesicles that have been induced by the co-overexpression of the glycoprotein VSV-G with, for example, a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene and regulatory sequence. The use of such vesicles, also referred to as Gesicles, for the genetic modification of eukaryotic cells is described in detail, for example, in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy, 2015 May 13, Abstract No. 122.

### AAV Vectors for Polynucleotide Delivery

The modified rep-cap plasmids described herein can be used to incorporate polynucleotides (for example, polynucleotides contained in a transgene plasmid) into rAAV (for example, rAAV1) vectors and/or virions in order to facilitate their introduction into a cell. According to the present inventions, rAAV vectors (for example, rAAV1) vectors produced using the modified rep-cap plasmids described herein are recombinant polynucleotide constructs that can include (1) a promoter, (2) a sequence to be expressed (for example, a polynucleotide encoding a protein or an RNA molecule), and (3) viral sequences that facilitate integration and expression of the sequence to be expressed. The viral sequences can include those sequences of AAV that are required in cis for replication and packaging (for example, functional ITRs) of the DNA into a virion. The sequence to be expressed can be a sequence encoding a RNA molecule or protein that is endogenously expressed in a cell that can be transduced with an AAV1 vector (for example, a RNA molecule or protein that is endogenously expressed in a cell of the central nervous system, heart, skeletal muscle, retinal pigment epithelium, or inner ear) or a sequence encoding a RNA molecule or protein of interest suitable for expression in a cell that can be transduced with an AAV1 vector (for example, a RNA molecule or protein intended to have a therapeutic effect in a cell of interest or to investigate the biology of the cell of interest). The sequence to be expressed can encode a protein that is endogenously expressed in the human ear.

The transgene sequence to be expressed can encode any one of solute carrier family 26, member 4 (Pendrin), Otoferlin (OTOF), Stereocilin (STRC), Atonal BHLH Transcription Factor 1 (ATOH1), gap junction protein beta 2 (GJB2), and SRY-Box 2 (Sox2). The sequence to be expressed can encode Pendrin, for example. The sequence to be expressed can encode GJB2, for example. The sequence to be expressed can encode OTOF, for example. The sequence to be expressed can encode STRC, for example. The sequence to be expressed can encode Atonal BHLH Transcription Factor 1 (ATOH1), for example. The sequence to be expressed can encode SRY-Box 2 (Sox2), for example.

Other transgenes that can be expressed include, for example, GJB6; WFS1; COCH; EYA4; MY07A; POU4F3; ACTG1; MY06; REST; NLRP3; COL11A1; TJP2; TBC1D24; SLC26A4; ELMOD3; EPSN; WHRN; IGF1; IGF1R; MYO15A; TMIE; TMC1; TMC2; TMPRSS3; CDH23; GIPC3; STRC; USH1C; OTOG; TECTA; OTOA; PDCH15; CLDN14; WHRN; ESRRB; MYO3A; HGF, ILDR1, ADCY1; CIB2; MARVELD2; SOX2; SLC26A5; COL4A3; COL4A4; COL4A5; CLPP; PJVK; LRTOMT/COMT2; LOXHD1; TPRM; SYNE4; KCNJ10; PTPRQ; OTOGL; LHFPL5; A1PR2; CABP2; MET; GRXCR2; EPS8; CLIC5; EPS8L2; WBP2; ROR1; CLDN9; USH1G; PKHD1L1; DIAPH3; NDP; USH2A; CLRN1; SANS; HARS1; TRIOBP. A transgene of interest includes any gene that is desired to be expressed and can fit within an AAV capsid. More than one transgene of interest is possible if the totality of genes are of a combined size that can fit within an AAV capsid.

Such rAAV vectors can also contain marker or reporter genes. Useful rAAV vectors can have one or more of the AAV WT genes deleted in whole or in part but retain functional flanking ITR sequences. The AAV ITRs can be of any serotype suitable for a particular application. The ITRs can be AAV2 ITRs. Methods for using rAAV vectors are described, for example, in Pupo et al., Mol Ther. 7:3515-3541 (2022), and Wang et al., Nat Rev Drug Discov. 18:358-378 (2019).

A polynucleotide (for example, a polynucleotide contained in a transgene plasmid that is co-transfected into a cell with a helper plasmid and a modified rep-cap plasmid described herein) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003).

### Manufacturing

Production of rAAV vectors for gene therapy can be carried out in vitro using suitable producer cell lines. Producer cells can be any cell type possessing the genes necessary to promote AAV genome replication, capsid assembly, and packaging. Exemplary producer cells can include human embryonic kidney 293 (HEK-293) cells or derivatives thereof, Hela cells, human amniotic cells, CHO cells, BHK cells, insect cells and others. One strategy for delivering all of the required elements for rAAV production can utilize two plasmids (for example, a modified rep-cap plasmid described herein and a transgene plasmid) and a helper virus. This method relies on transfection of the producer cells with plasmids containing gene cassettes encoding the necessary gene products, as well as infection of the cells with adenovirus to provide the helper functions. This system employs plasmids with two different gene cassettes. The first can be a proviral plasmid encoding the recombinant DNA to be packaged as rAAV (that is, the transgene plasmid). The second can be a plasmid containing polynucleotides encoding the rep and capsid proteins (for example, a modified rep-cap plasmid described herein). To introduce these various elements into the cells, the cells can be infected with adenovirus as well as transfected with the two plasmids. Alternatively, the adenovirus infection step can be replaced by transfection with a helper plasmid containing the VA, E2A and E4 genes. Another alternative approach can involve incorporating the helper genes (for example, VA, E2A, and/or E4) into the rep-cap plasmid (for example, into a modified rep-cap plasmid described herein) so only two plasmids are needed to produce an rAAV vector.

While adenovirus has been used conventionally as the helper virus for rAAV production, it is known that other DNA viruses, such as Herpes simplex virus type 1 (HSV-1) can be used as well. The minimal set of HSV-1 genes required for AAV2 replication and packaging has been identified, and includes the early genes UL5, UL8, UL52 and UL29. These genes encode components of the HSV-1 core replication machinery, that is, the helicase, primase, primase accessory proteins, and the single-stranded DNA binding protein. This rAAV helper property of HSV-1 has been utilized in the design and construction of a recombinant Herpes virus vector capable of providing helper virus gene products needed for rAAV production.

The production of an rAAV1 vector using a modified rep-cap plasmid described herein can result in an increased yield of rAAV1 virus compared to production of rAAV1 virus using an unmodified rep-cap plasmid containing a polynucleotide encoding an AAV2 rep protein and a polynucleotide encoding an AAV1 capsid protein and lacking a tetracycline-inducible expression system (for example, the pAAV-RC1 vector produced by Cell Biolabs, Inc.). For example, production of an rAAV1 vector using a modified rep-cap plasmid described herein can result in a yield of rAAV1 virus that can be increased by at least 2.5-fold (for example, 2.5-fold, 2.7-fold, 3.0-fold, 3.1-fold, 3.5-fold, 4-fold, 4.5-fold, 5-fold, 5.5-fold, 6-fold, 6.5-fold, 7-fold, 8-fold, 9-fold, 10-fold, 11-fold, 12-fold, 13-fold, 14-fold, 15-fold, 20-fold, or more) compared to production of an rAAV1 vector using an unmodified AAV2 rep-AAV1 cap plasmid (for example, pAAV-RC1). The yield can be increased by at least 7-fold (for example, 7-fold, 8-fold, 9-fold, 10-fold, 15-fold, 20-fold, or more). The production of an rAAV1 virus using a modified rep-cap plasmid described herein (for example, by introducing the modified rep-cap plasmid into a cell with a transgene plasmid and a helper plasmid) can result in a viral yield of at least 1×10¹¹ vg/mL (for example, 1×10¹¹ vg/mL, 2×10¹¹ vg/mL, 3×10¹¹ vg/mL, 4×10¹¹ vg/mL, 5×10¹¹ vg/mL, or greater, such as such as 1×10¹¹ vg/mL to 5×10¹¹ vg/mL, 1×10¹¹ vg/mL to 1×10¹² vg/mL, 1×10¹¹ vg/mL to 5×10¹² vg/mL, or 1×10¹¹ vg/mL to 1×10¹³ vg/mL) as determined by ddPCR of clarified lysate or a viral yield of at least 1×10¹³ vg/mL (for example, 1×10¹³ vg/mL, 2×10¹³ vg/mL, 3×10¹³ vg/mL, 4×10¹³ vg/mL, 5×10¹³ vg/mL, or greater, such as such as 1×10¹³ vg/mL to 5×10¹³ vg/mL, 1×10¹³ vg/mL to 1×10¹⁴ vg/mL, 1×10¹³ vg/mL to 5×10¹⁴ vg/mL, or 1×10¹³ vg/mL to 1×10¹⁵ vg/mL) as determined by ddPCR of purified vector. An increased rAAV1 production can be achieved when the tetracycline-inducible expression system is active (for example, by producing rAAV1 using a modified rep-cap plasmid containing a Tet-Off system in the absence of an inducer of the Tet-Off system or by producing rAAV1 using a modified rep-cap plasmid containing a Tet-On system in the presence of an inducer of the Tet-On system). Also, an increased rAAV1 production can be achieved when the tetracycline-inducible expression system is “inactive” (for example, by producing rAAV1 using a modified rep-cap plasmid containing a Tet-Off system in the presence of an inducer of the Tet-Off system or by producing rAAV1 using a modified rep-cap plasmid containing a Tet-On system in the absence of an inducer of the Tet-On system). The tetracycline-inducible expression systems contained in the modified rep-cap plasmids described herein can be leaky, meaning that some rAAV1 production still occurs even when the tetracycline-inducible expression system is “inactivated.”

The production of an rAAV1 vector using a modified rep-cap plasmid described herein under conditions in which the tetracycline-inducible expression system is “inactive” (for example, producing rAAV1 using a modified rep-cap plasmid containing a Tet-Off system in the presence of an inducer of the Tet-Off system or producing rAAV1 using a modified rep-cap plasmid containing a Tet-On system in the absence of an inducer of the Tet-On system) can result in an increased percent of full AAV1 vectors compared to the production of full AAV1 vectors using the same rep-cap plasmid under conditions in which the tetracycline-inducible expression system is “active” (for example, producing rAAV1 using a modified rep-cap plasmid containing a Tet-Off system in the absence of an inducer of the Tet-Off system or producing rAAV1 using a modified rep-cap plasmid containing a Tet-On system in the presence of an inducer of the Tet-On system). The rAAV1 production using a rep-cap plasmid described herein under “inactivating” conditions can increase the production of full rAAV1 vectors by at least 1, 2, 3, 4, 5, 6, 7, 8, 9, 10 or more percentage points (for example, from 4% full to 6%, 8%, 10%, 12%, 14% full, or more, or from 10% full to 12%, 14%, 16%, 18%, 20%, 22% full, or more).

The production of an rAAV1 virus using an uninduced (that is, “inactive,” for example, without added doxycycline or any other inducer) Tet-On modified rep-cap plasmid described herein (for example, by introducing the modified rep-cap plasmid described herein into a cell with a transgene plasmid and a helper plasmid) can result in a viral yield of at least 1×10¹¹ vg/mL (for example, 1×10¹¹ vg/mL, 2×10¹¹ vg/mL, 3×10¹¹ vg/mL, 4×10¹¹ vg/mL, 5×10¹¹ vg/mL, or greater, such as such as 1×10¹¹ vg/mL to 5×10¹¹ vg/mL, 1×10¹¹ vg/mL to 1×10¹² vg/mL, 1×10¹¹ vg/mL to 5×10¹² vg/mL, or 1×10¹¹ vg/mL to 1×10¹³ vg/mL) as determined by ddPCR of clarified lysate or a viral yield of at least 1×10¹³ vg/mL (for example, 1×10¹³ vg/mL, 2×10¹³ vg/mL, 3×10¹³ vg/mL, 4×10¹³ vg/mL, 5×10¹³ vg/mL, or greater, such as such as 1×10¹³ vg/mL to 5×10¹³ vg/mL, 1×10¹³ vg/mL to 1×10¹⁴ vg/mL, 1×10¹³ vg/mL to 5×10¹⁴ vg/mL, or 1×10¹³ vg/mL to 1×10¹⁵ vg/mL) as measured by ddPCR in clarified lysate and at least an estimated percent of full AAV1 vectors (AAV1 vectors that also contain the transgene) of at least 20% (for example, 20%, 21%, 22%, 23%, 24%, 25%, 26%, 27%, 28%, 29%, 30%, or more) as determined using SEC-MALS.

While the rep-cap plasmids described herein can include a polynucleotide encoding an AAV1 capsid protein and are for use in the manufacture of rAAV1 vectors, the modified rep-cap plasmids can be used to produce rAAV vectors having a different capsid if the polynucleotide encoding an AAV1 capsid protein is replaced with a polynucleotide encoding a different AAV capsid protein. For example, the polynucleotide encoding an AAV1 capsid protein can be replaced with a polynucleotide encoding an AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP.S capsid protein in any of the rep-cap plasmids described herein. Such rep-cap plasmids can be used to produce AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP.S vectors using the methods described herein (for example, by introducing the rep-cap plasmid into a producer cell along with a transgene plasmid and helper plasmid). According to the current inventions, AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP. S vectors can be produced using these modified rep-cap plasmids by manufacturing the rAAV vectors in conditions that would cause the tetracycline-inducible expression system to be “inactive” (for example, producing AAV vectors using a modified rep-cap plasmid that contains a Tet-Off system in the presence of an inducer of the Tet-Off system, or producing AAV vectors using a modified rep-cap plasmid that contains a Tet-On system in the absence of an inducer of the Tet-On system). The manufacturing of AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP.S vectors using modified rep-cap plasmids that contain a polynucleotide encoding an AAV2, AAV2quad (Y-F), AAV3, AAV4, AAV5, AAV6, AAV7, AAV8, AAV9, AAV10, AAV11, AAV12, AAV13, rh10, rh39, rh43, rh74, Anc80, Anc80L65, DJ, DJ/8, DJ/9, 7m8, PHP.B, PHP.eB, or PHP.S capsid protein in “inactivating” conditions can lead to a higher percentage of full vectors than manufacturing of rAAV vectors using the same rep-cap plasmids in “activating” conditions (for example, conditions in which the tetracycline-inducible expression system would be turned “on,” such as producing AAV vectors using a modified rep-cap plasmid that contains a Tet-Off system in the absence of an inducer of the Tet-Off system, or producing AAV vectors using a modified rep-cap plasmid that contains a Tet-On system in the presence of an inducer of the Tet-On system).

Recombinant AAV vectors (for example, AAV1 virus) produced using a modified rep-cap plasmid described herein can be purified by any suitable purification method known in the art, such as, for example, chromatography, ultra-centrifugation, centrifugation, flocculation, filtration, and/or ultrafiltration/diafiltration. For example, the rAAV1 virus can be purified by chromatography. The rAAV1 virus can be purified by ultra-centrifugation. The rAAV1 virus can be purified by centrifugation. The rAAV1 virus can be purified by flocculation. The rAAV1 virus can be purified by filtration. The rAAV1 virus also can be purified by ultrafiltration/diafiltration.

### Kits

The modified rep-cap plasmids described herein can be provided in a kit. The kit can include a modified rep-cap plasmid and a helper plasmid, or a modified rep-cap plasmid, a helper plasmid, and a transgene plasmid. The kit also can include a package insert that instructs a user of the kit, such as a scientist of skill in the art, to perform any one of the methods described herein. For example, the kit can include a package insert that instructs a user of the kit to manufacture an rAAV vector (for example, an rAAV1 vector) using a modified rep-cap plasmid described herein.

### EXAMPLES

The following examples are put forth so as to provide those of ordinary skill in the art with a description of how the compositions and methods described herein may be used, made, and evaluated, and are intended to be purely exemplary of the inventions and are not intended to limit the scope of what the inventors regard as their inventions.

### Example 1. Comparing AAV1 Production Using Standard and Modified Rep-Cap Plasmids

### AAV Production

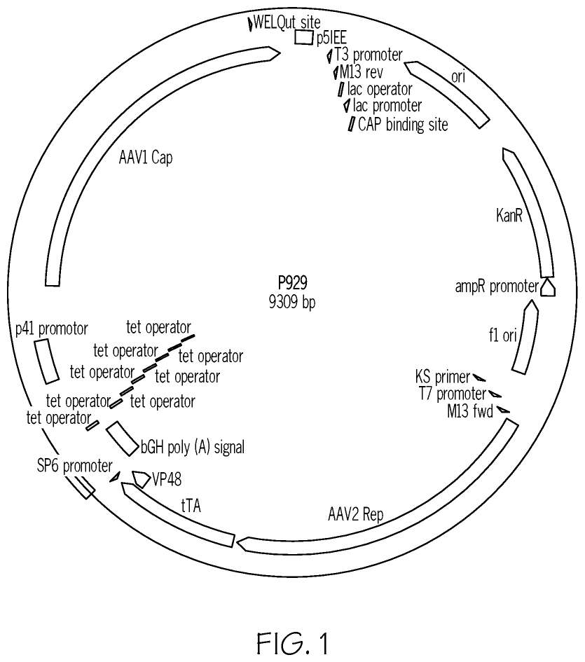

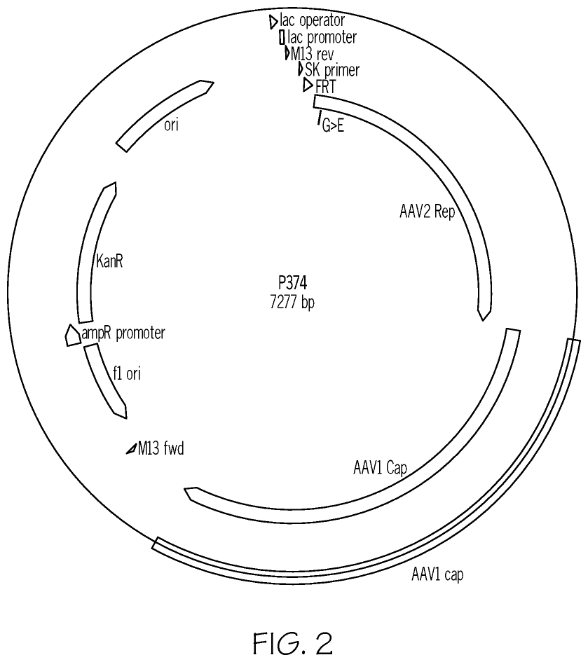

To enable AAV production, AAV2 rep/AAV1 cap encoding plasmid, pHelper plasmid, and transfer plasmid at a 2:1:2 concentration ratio were combined with FectoVIR (Polypus) at a 1:1 FectoVIR: DNA (deoxyribonucleic acid) ratio and added to suspension VPC (viral production cells, 293 origin) cultures. The AAV2 rep/AAV1 cap encoding plasmid used for AAV production was either modified rep-cap plasmid P929 (FIG. 1) or standard rep-cap plasmid P374 (FIG. 2). P929 was developed according to the inventions to provide AAV1 rep/cap in PHP. B tet-off backbone. P929 contains 2 silent single point mutations in the CAP open reading frame for VP1:2:3 (may not be silent in AAP ORF) compared to P374, considered to have the wild-type AAV1 CAP sequence.

The transfer plasmids used for AAV production were transfer plasmid P1381, which contained a CMV promoter operably linked to a polynucleotide encoding enhanced GFP, transfer plasmid P1900, which contained a pendrin promoter operably linked to a polynucleotide encoding pendrin, and transfer plasmid P707, which contained a CMV promoter operably linked to a polynucleotide encoding GFP. The same pHelper plasmid (P376) was used with all rep-cap plasmid and transfer plasmid combinations. The cell culture medium and cells were harvested after 72 hours of incubation at 37° C., 8% CO₂, and 125 RPM. The cell lysate was then treated with benzonase nuclease to degrade nonviral DNA and RNA (ribonucleic acid) and Triton X-100 (10% Triton X-100 w/v) and lysed during incubation at 37° C., 8% CO₂, and 125 RPM. Cell lysates were then filtered through a 0.22 μm filter prior to purification.

AAV particles were enriched from the cell culture lysate via gravity affinity chromatography using AAVX resin, and subsequently buffer exchanged using centrifugal filtration units. The AAV was formulated in 10 mM Sodium Phosphate, 180 mM NaCl, 5% Sucrose (w/v) and 0.001% poloxamer 188 (w/v), pH 7.3 and stored at −80° C. The resulting AAV and their transfer plasmid sources are indicated in Table 2.

TABLE 2
Content of AAV particles

| Sample | Rep/Cap | pHelper | GOI (encoded protein) |
|---|---|---|---|
| AAV1439 | P929 | P376 | P1381 (GFP) |
| AAV1441 | P929 | P376 | P1900 (pendrin) |
| AAV1440 | P929 | P376 | P707 (GFP) |
| AAV1442 | P374 | P376 | P1381 (GFP) |
| AAV1444 | P374 | P376 | P1900 (pendrin) |
| AAV1443 | P374 | P376 | P707 (GFP) |

### Vector Genome Titer by Droplet Digital Polymerase Chain Reaction

Droplet digital polymerase chain reaction (ddPCR) was used to quantify the total number of vector genomes within the AAV. The ddPCR method employed a set of primers and probe specific for the gene of interest. The primers/probe set used for samples AAV1439, AAV1440, AAV1442, and AAV1443, were directed to the Woodchuck Hepatitis Virus Posttranscriptional Regulatory Element (WPRE), a sequence present in all four viruses. Primer and probe sequences included forward primer: TTGTGAAAGATTGACTGGTATTCT (SEQ ID NO: 16), reverse primer: AGGCATTAAAGCAGCGTA (SEQ ID NO: 17), and probe: TAACTATGTTGCTCCTTTTAC (SEQ ID NO: 18). For the remaining two samples (AAV1441 and AAV1444), the primers/probe included in the Predesigned TaqMan Gene Expression Assay Hs01070627_m1 from ThermoFisher were used. This proprietary set specifically is directed to a sequence in the SLC26A4 gene-which encodes the pendrin protein-included in the two viruses. The sequence is:

(SEQ ID NO: 19)
AAGGGATGGCATATGCCCTACTAGCTGCAGTTCCTGTCGGATATGGTCT
CTACTCTGCTTTTTTCCCTATCC

To perform the ddPCR test, a DNAse digestion (30 min at 37° C.) was performed to remove all non-encapsidated DNA and ensure that only endonuclease-resistant (encapsidated) vector genomes are quantitated. This step used recombinant DNase I from Sigma-Aldrich (10 units/μL, catalog number 4716728001). Next, a proteinase K (ProK, >600 mAU/mL, Qiagen catalog number 19133) treatment was used to neutralize DNAse and digest the AAV capsid to release any encapsidated DNA, followed by a heat treatment to inactivate the enzymes and open the capsid. Incubation parameters were: 56° C. for 30 minutes, 95° C. for 5 minutes, and 4° C. for 5 minutes to 1 hour. The sample was then diluted, and polymerase chain reaction (PCR) master mix, containing Bio-Rad PCR Supermix (1863023) plus primer and probe sets, was added.

For the ddPCR assay, the PCR mix (test sample, master mix with primers, and probe) was partitioned into nanoliter-scale droplets through a water-oil emulsion system using the Bio-Rad Automatic Droplet Generator. The test sample was randomly partitioned into the droplets and amplified through PCR using a Bio-Rad C1000 Touch Thermal Cycler. At the end of the PCR reaction, the fluorescence in each droplet was individually measured with the Bio-Rad QX200 Droplet Reader. The internal probe was labeled with a 5′ fluorescent reporter dye and a non-fluorescent quencher on the 3′ end. With each PCR cycle, the Taq polymerase 5′ exonuclease activity hydrolyzed the internal probe, releasing fluorophore from the probe and quencher. Each droplet was assigned as negative (no measurable DNA) or positive (one or more copies of DNA) based on a fluorescence intensity threshold, which was determined from the no-template control. The absolute concentration of DNA in each sample was determined according to the Poisson distribution, based on the proportions of positive to negative droplets for different dilutions of the test sample. The dilution-corrected concentration of the gene of interest (GOI) was reported in units of vg/mL.

As shown in Table 3, production of AAV1 using modified rep-cap plasmid P929 resulted in increased titer compared to production of AAV1 using standard rep-cap plasmid P374. For example, comparisons of clarified lysate ddPCR titer (vg/mL) showed a 13.9-fold difference between the vector genome titer of AAV1 produced with modified rep-cap plasmid P929 and transfer plasmid P1381 versus standard rep-cap plasmid P374 and transfer plasmid P1381, a 13.2-fold difference between the vector genome titer of AAV1 produced with modified rep-cap plasmid P929 and transfer plasmid P1900 versus standard rep-cap plasmid P374 and transfer plasmid P1900, and a 9.1-fold difference between the vector genome titer of AAV1 produced with modified rep-cap plasmid P929 and transfer plasmid P707 versus standard rep-cap plasmid P374 and transfer plasmid P707. Comparisons of purified ddPCR titer (vg/mL) also showed an increase in titer of AAV1 produced using modified rep-cap plasmid P929 versus AAV1 produced using standard rep-cap plasmid P374, with a 16.5-fold difference for AAV1 produced with transfer plasmid P1381, a 15.2-fold difference for AAV1 produced with transfer plasmid P1900, and a 68.89-fold difference for AAV1 produced with transfer plasmid P707.

### Vector Capsid Titer by Enzyme-Linked Immunosorbent Assay

A sandwich enzyme-linked immunosorbent assay (ELISA) was used to quantitate the total capsid content in all samples.

The AAV1 capsid assay was performed as a two-step colorimetric ELISA using commercially available capture and detection reagents, calibration standards, and assay diluent. Two different versions of the same single-chain camelid antibody, which binds with high affinity to AAV capsids, were used. The capture antibody (ThermoFisher 7103522100, 1 mg/mL) was the anti-AAV antibody conjugated to biotin; the detection antibody (ThermoFisher 7303522100, 0.5 mg/mL) was the same anti-AAV antibody conjugated to Horseradish Peroxidase (HRP).

Each well of streptavidin-coated plates (ThermoFisher number 15124) was incubated for one hour at room temperature with 100 ng of the capture biotin-conjugate antibody. After washing, calibration standards and diluted test samples containing AAV1 capsids were added to the wells of the plate and incubated for one hour at room temperature. The standard curve consisted of 2.5 serial dilutions of empty AAV1 capsids (Vigene RS-AAV1-ET), ranging from 1.00×10¹¹ to 1.64×10⁸ capsids/mL. The plates were washed, and 100 μL of the detection HRP-conjugate antibody, diluted 1:10,000, was added the wells and incubated for one hour at room temperature. Following washing, 3,3′,5,5′-tetramethylbenzidine (TMB) substrate (ThermoFisher 34028) was added to the wells of the plate. After ten minutes, the reaction was stopped by adding 25% sulfuric acid (Sigma-Aldrich 84736-1L), resulting in a colorimetric reaction that was proportional to the concentration of AAV1 capsids in the calibration standards and diluted test samples. Absorbance was measured on a PerkinElmer EnVision Multimode Plate Reader at 450 nm. Concentrations were determined by interpolation from a standard curve fit with a 4-parameter logistic (4PL) model using GraphPad Prism. Triplicate measurements were performed for each standard, assay control, and test sample. The dilution-corrected concentration of AAV1 capsids was reported in units of capsids/mL.

As shown in Table 3, production of AAV1 using modified rep-cap plasmid P929 resulted in increased capsid titer (cp/mL) and total capsids compared to production of AAV1 using standard rep-cap plasmid P374. For example, comparisons of clarified lysate capsid titer (cp/mL) showed a 18.8-fold difference between the capsid titer of AAV1 produced with modified rep-cap plasmid P929 and transfer plasmid P1381 versus standard rep-cap plasmid P374 and transfer plasmid P1381, a 15.8-fold difference between the capsid titer of AAV1 produced with modified rep-cap plasmid P929 and transfer plasmid P1900 versus standard rep-cap plasmid P374 and transfer plasmid P1900, and a 27.2-fold difference between the capsid titer of AAV1 produced with modified rep-cap plasmid P929 and transfer plasmid P707 versus standard rep-cap plasmid P374 and transfer plasmid P707.

TABLE 3
Vector Production using Standard and Tet-Off Modified Rep-Cap Plasmids

| | Clarified | Total | Estimated | Clarified | Estimated | Estimated | |
|---|---|---|---|---|---|---|---|
| | Lysate | Capsids | ddPCR | lysate | % Full | Purified | Purified |
| | Capsid | (per | titer | ddPCR | based on | ddPCR | ddPCR |
| | Titer | 120 mL | (based on | Actual | ratio of | Titer | Titer |
| Sample | (cp/mL) | lysate) | 25% full) | (vg/mL) | VG/CP | (vg/mL) | (vg/mL) |
| AAV1439 | 1.45E+12 | 1.74E+14 | 3.63E+11 | 3.03E+11 | 21% | 3.64E+13 | 4.20E+13 |
| AAV1441 | 1.22E+12 | 1.46E+14 | 3.05E+11 | 3.88E+11 | 32% | 4.66E+13 | 3.36E+13 |
| AAV1440 | 7.30E+11 | 8.76E+13 | 1.83E+11 | 1.10E+11 | 15% | 1.32E+13 | 2.26E+13 |
| AAV1442 | 7.68E+10 | 9.22E+12 | 1.92E+10 | 2.18E+10 | 28% | 2.61E+12 | 2.54E+12 |
| AAV1444 | 7.69E+10 | 9.23E+12 | 1.92E+10 | 2.94E+10 | 38% | 3.53E+12 | 2.20E+12 |
| AAV1443 | 2.68E+10 | 3.22E+12 | 6.70E+09 | 1.20E+10 | 45% | 1.45E+12 | 3.28E+11 |

### Example 2. Comparing AAV1 Production Using Standard and Modified Rep-Cap Plasmids

### AAV Production

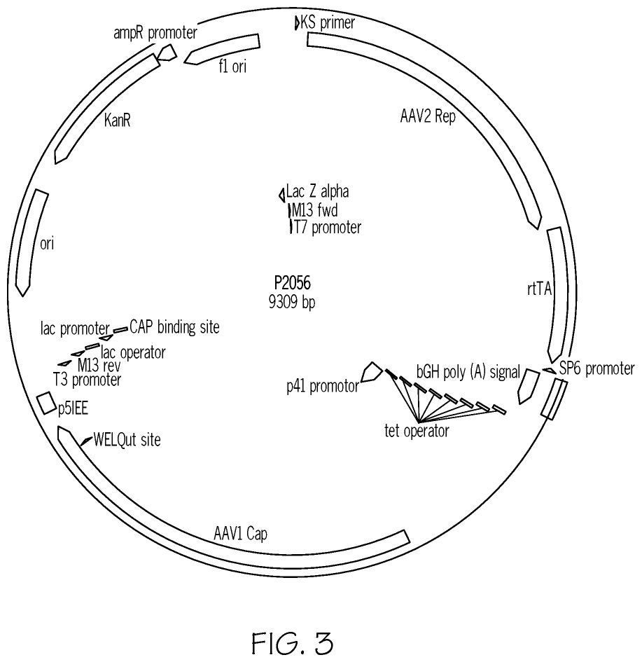

To enable AAV production, AAV2 rep/AAV1 cap encoding plasmid, pHelper plasmid, and transfer plasmid, at a 2:1:2 concentration ratio were combined with FectoVIR (Polypus) at a 1:1 FectoVIR: DNA (deoxyribonucleic acid) ratio and added to suspension VPC (viral production cells, 293 origin) cultures. The AAV2 rep/AAV1 cap encoding plasmid used for AAV production was either modified rep-cap plasmid P2056 (FIG. 3) or standard rep-cap plasmid P374 (FIG. 2). The transfer plasmid used for AAV production was transfer plasmid P1381, which contained a CMV promoter operably linked to a polynucleotide encoding GFP. The same pHelper plasmid (P376) was used with each rep-cap plasmid and transfer plasmid combination. The P2056 rep/cap plasmid contains a rtTA inducible system that is induced by doxycycline. We tested three concentrations of doxycycline (1 μg/ml, 0.1 μg/ml, and 0.01 μg/m) added concurrently upon transfection with the P2056/P1381/P374 system, as well as an untreated control. We also tested the P376/P1381/P374 system, which does not contain a tetracycline-inducible rep/cap by adding 1 μg/ml doxycycline concurrently upon transfection to determine the impact of doxycycline on transfection. The cell culture medium and cells were harvested after 72 hours of incubation at 37° C., 8% CO₂, and 125 RPM. The cell lysate was then treated with benzonase nuclease to degrade nonviral DNA and RNA (ribonucleic acid) and Triton X-100 (10% Triton X-100 w/v) and lysed during incubation at 37° C., 8% CO₂, and 125 RPM. Cell lysates were then filtered through a 22 μm filter prior to purification.

AAV particles (AAV1556 for the P2056/P1381/P374 co-transfection; and AAV1442 for the P376/P1381/P374 co-transfection) were enriched from the cell culture lysate via gravity affinity chromatography using AAVX resin, and subsequently buffer exchanged using centrifugal filtration units. The AAV was formulated in 10 mM sodium phosphate, 180 mM NaCl, 5% sucrose (w/v) and 0.001% poloxamer 188 (w/v), pH 7.3 and stored at −80° C.

### Vector Genome Titer by Droplet Digital Polymerase Chain Reaction

Droplet digital polymerase chain reaction (ddPCR) was used to quantify the total number of vector genomes within the AAV and performed as described in Example 1 using the same forward and reverse primers (SEQ ID NOs: 16 and 17) and probe (SEQ ID NO: 18).

As shown in Table 5, production of AAV1 using modified rep-cap plasmid P2056 resulted in increased titer compared to production of AAV1 using standard rep-cap plasmid P374 as determined by ddPCR. For example, comparisons of clarified lysate ddPCR titer (vg/mL) showed a 2.7-fold difference between the vector genome titer of AAV1 produced with modified rep-cap plasmid P2056 versus standard rep-cap plasmid P374 in the absence of added doxycycline, and a 3.1-fold difference in the presence of 1.0 μg/ml doxycycline. Comparisons of purified ddPCR titer (vg/mL) also showed an increase in titer of AAV1 produced using modified rep-cap plasmid P2056 versus AAV1 produced using standard rep-cap plasmid P374, with a 5.0-fold difference for AAV1 produced in the absence of doxycycline and a 3.0-fold difference for AAV1 produced in the presence of 1 μg/ml doxycycline. Interestingly, the titer of AAV1 produced with modified rep-cap plasmid P2056 in both clarified lysate and following affinity purification was minimally affected by the presence or absence of added doxycycline as determined by ddPCR. We believe this is due to the fact that the Tet-On system employed in P2056 is leaky and allowed for capsid protein production even in the absence of added doxycycline.

### Vector Capsid and Genome Titer and Empty/Full Analysis by Size Exclusion Chromatography-Multi Angle Light Scattering

Size Exclusion Chromatography-Multi Angle Light Scattering (SEC-MALS) analysis was performed using an Agilent 1260 Infinity II HPLC instrument equipped with a Multiwavelength UV-Vis detector, and a Wyatt Dawn MALS detector. Separation was accomplished with a 7.8×300 mm Agilent Bio SEC-5 column, packed with 5 μm particles with 1000 Å pores, and a mobile phase containing 300 mM NaCl, 20 mM Na phosphate, pH 7.4, and 0.001% (w/v) Pluronic® F68, at a flow rate of 1.0 mL/min. The volume of each injection was 50 μL. Determination of capsid titers (cp/mL) and genome titers (vg/mL) was accomplished using the Viral Vector procedure, which is based on Conjugate Analysis, within the Astra software (version 8.1.2.1). The procedure, which is described in the Wyatt Technology Application Note 1617 (“Quantifying quality attributes of AAV gene therapy vectors by SEC-UV-MALS-dRI”) and discussed in Werle et al., Mol Ther Methods Clin Dev. 23:254-262, 2021 in comparison with other techniques used to determine AAV empty/full ratios, entails the use of signals from multi-angle light scattering together with two concentration sources, such as UV absorbance at 260 and 280 nm. These signals are used to calculate the total protein and DNA masses, and the capsid and transgene molar masses (Mw), which in turn are converted into capsid and genome titers. Finally, the % full capsids is determined by the following formula: (capsid titer)/(genome titer)×100. The processing parameters that were used for the SEC-MALS analysis are listed in Table 4.

TABLE 4
Parameters used for SEC-MALS analysis

| Parameter | Value |
|---|---|
| Protein Ext. Coeff. at 280 nm (mL mg⁻¹ cm⁻¹) | 2.10 |
| Protein Ext. Coeff. at 260 nm (mL mg⁻¹ cm⁻¹) | 1.30 |
| Expected Protein Molar Mass (MDa) | 3.75 |
| Nucleic Acid Ext. Coeff. at 280 nm (mL mg⁻¹ | 14.6 |
| cm⁻¹) | |
| Nucleic Acid Ext. Coeff. at 260 nm (mL mg⁻¹ | 24.0 |
| cm⁻¹) | |
| Expected Nucleic Acid Molar Mass (MDa) | 0.94 |

The results of the SEC-MALS analysis are shown in Table 5. As measured by SEC-MALS, production of AAV1 using modified rep-cap plasmid P2056 resulted in a 4.7-fold increase in purified viral titer compared to production of AAV1 using standard rep-cap plasmid P374 in the absence of doxycycline, and a 3.5-fold increase in the presence of 1.0 μg/ml doxycycline. Capsid titer using P2056 was also increased by 4.1-fold in the absence of doxycycline and 6.7-fold in the presence of 1.0 μg/ml doxycycline, respectively over P374. The SEC-MALS results also confirmed that doxycycline had minimal effect on purified viral titer using modified rep-cap plasmid P2056. Surprisingly, the production of full AAV1 vectors using P2056 was higher in the absence of added doxycycline (23.1%) than in its presence (10.1-18.5%).

TABLE 5
Vector Production using Standard and Tet-On Modified Rep-Cap Plasmids

| | | | | | | SEC- | SEC- | % Full |
|---|---|---|---|---|---|---|---|---|
| | | | | ddPCR | ddPCR | MALS | MALS | based |
| | | | | (vg/mL) | (vg/mL) | Purified | Capsid | on |
| | Rep/Cap | Doxycycline | | Clarified | Affinity | Titer | Titer | SEC- |
| Sample | Plasmid | Added | | Lysate | Purified | (vg/ml) | (cp/mL) | MALS |
| AAV1556 | P2056 | 1.0 | μg/mL | 1.01E+11 | 1.10E+13 | 8.06E+12 | 7.96E+13 | 10.1 |
| AAV1556 | P2056 | 0.1 | μg/mL | 8.14E+10 | 8.89E+12 | 6.42E+12 | 5.86E+13 | 11 |
| AAV1556 | P2056 | 0.01 | μg/mL | 9.96E+10 | 1.25E+13 | 9.02E+12 | 4.88E+13 | 18.5 |
| AAV1556 | P2056 | 0 | | 1.05E+11 | 1.51E+13 | 1.09E+13 | 4.71E+13 | 23.1 |
| AAV1442 | P374 | 1.0 | μg/mL | 3.30E+10 | 3.61E+12 | 2.29E+12 | 1.18E+13 | 19.4 |

| | | | | | | | |
|---|---|---|---|---|---|---|---|
| AAV1442 | P374 | 0 | 3.95E+10 | 3.03E+12 | 2.33E+12 | 1.16E+13 | 20.1 |

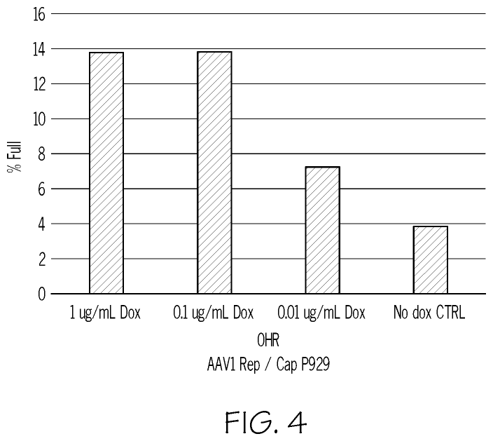

In another experiment, modified rep-cap plasmid P929 was used to produce AAV1 in the presence of 1.0 μg/ml doxycycline, 0.1 μg/ml doxycycline, or 0.01 μg/ml doxycycline, or in the absence of doxycycline. The percentage of full AAV1 vectors was assessed using SEC-MALS using a similar method to that described above. Surprisingly, the production of full AAV1 vectors using P929 was higher in the presence of added doxycycline than in its absence (FIG. 4).

### Example 3. Comparing Production of AAV1 Encoding the GJB2 Transgene Using Standard and Modified Rep-Cap Plasmid

The GJB2 transgene encodes the connexin 26 protein. Mutations in this gene are implicated in hearing loss.

A comparison was run between an AAV-pRC1 variant and P929 (See FIG. 1) in the production of AAV1 encoding the GJB2 transgene. The AAV-pRC1 variant, termed RC1 p5AS, was used as a control. Production was undertaken in the presence of 1.5 μg/ml doxycycline or in the absence of doxycycline. The same pHelper plasmid (P376) was used.

The productions were conducted in 125 mL shake flasks with 30 mL working volume. After 72 hours of incubation at 37° C., 8% CO2, and 120 RPM, the cell culture was lysed using 1% Tween 20 and 100 U/mL Denarase nuclease. Cell lysates were clarified via centrifugation prior to genomic and capsid titer quantification.

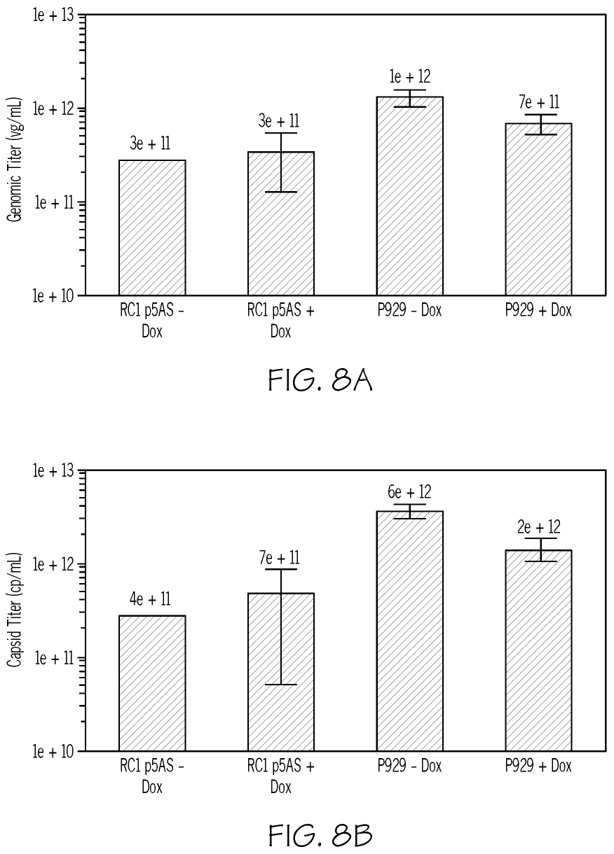

FIGS. 8A and 8B depict data from production of AAV1 containing a GJB2 transgene using an RC51 p5AS or P929. FIG. 8A depicts genomic titer and shows that P929 was superior to RC1 p5AS in the presence of doxycycline (over 2 fold superior) and the absence of doxycycline (over 3 fold superior). FIG. 8B depicts capsid titer and shows that P929 was superior to RC1 p5AS in the presence of doxycycline (almost 3 fold superior) and the absence of doxycycline (over 10 fold superior). Table 6 below sets forth the tabulated data.

TABLE 6
Tabulated Data for P929 Compared to RC1 p5AS

| | | | Clarified | Clarified Lysate |
|---|---|---|---|---|
| | Rep/Cap | Doxycycline | Lysate Capsid | Genomic Titer |
| Sample | Plasmid | Added | Titer (cp/mL) | (vg/mL) |
| CER5631 | RC1 p5AS | 0 | 3.63E+11 | 2.94E+11 |
| CER5630 | RC1 p5AS | 1.5 ug/mL | 2.45E+11 | 1.90E+11 |
| CER5637 | RC1 p5AS | 1.5 ug/mL | 1.14E+12 | 4.85E+11 |
| CER5629 | P929 | 0 | 5.65E+12 | 1.17E+12 |
| CER5635 | P929 | 0 | 7.18E+12 | 1.50E+12 |
| CER5628 | P929 | 1.5 ug/mL | 1.78E+12 | 5.80E+11 |
| CER5634 | P929 | 1.5 ug/mL | 2.83E+12 | 8.15E+11 |

### Example 4. AAV1 Expressing the Enhanced Green Fluorescent Protein Transgene

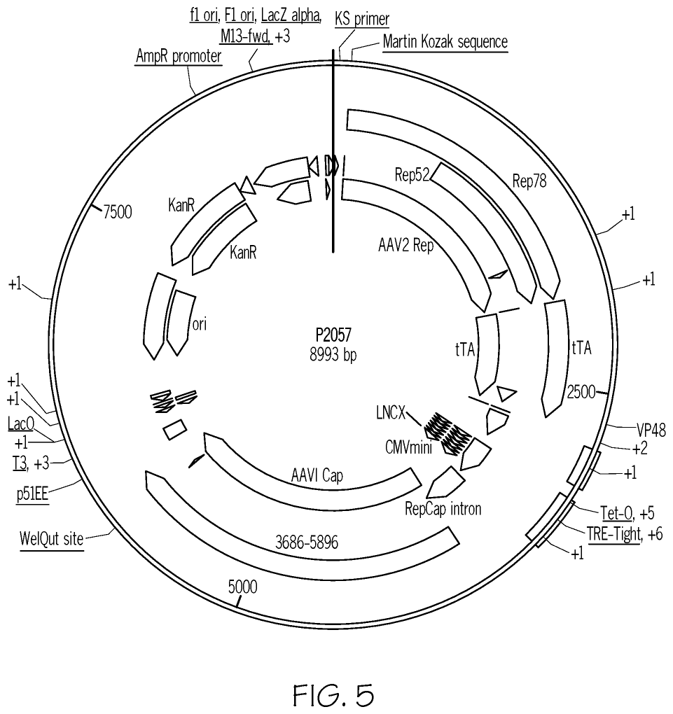

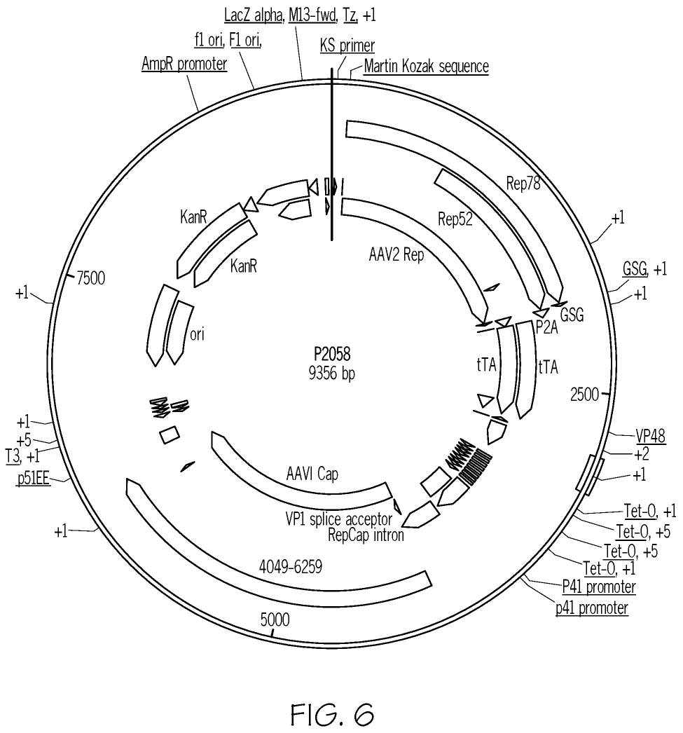

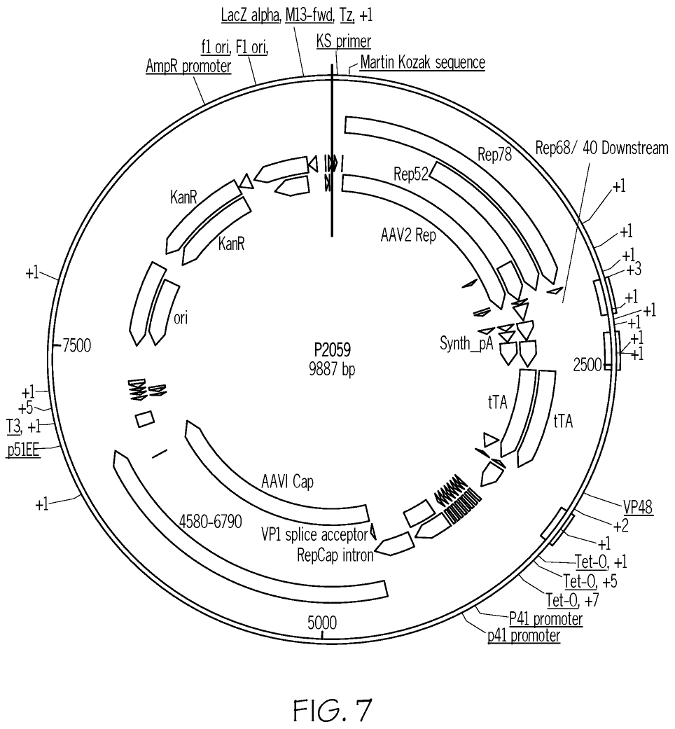

This example compares plasmids P374 (FIG. 2), P929 (FIG. 1), P2057 (FIG. 5), P2058 (FIG. 6) and P2059 (FIG. 7).

Plasmid 2057 is based upon P929 with the promoter changed for a TRE-tight promoter. Plasmid 2058 has the tTA (tetracycline transactivator) linked to the Rep gene by the 2A peptide. The 2A peptide is an 18-22 amino-acid peptide that mediates self-cleavage of polypeptides during translation in eukaryotic cells. Plasmid 2059 has tTA regulated under its own promoter and not regulated by Rep.

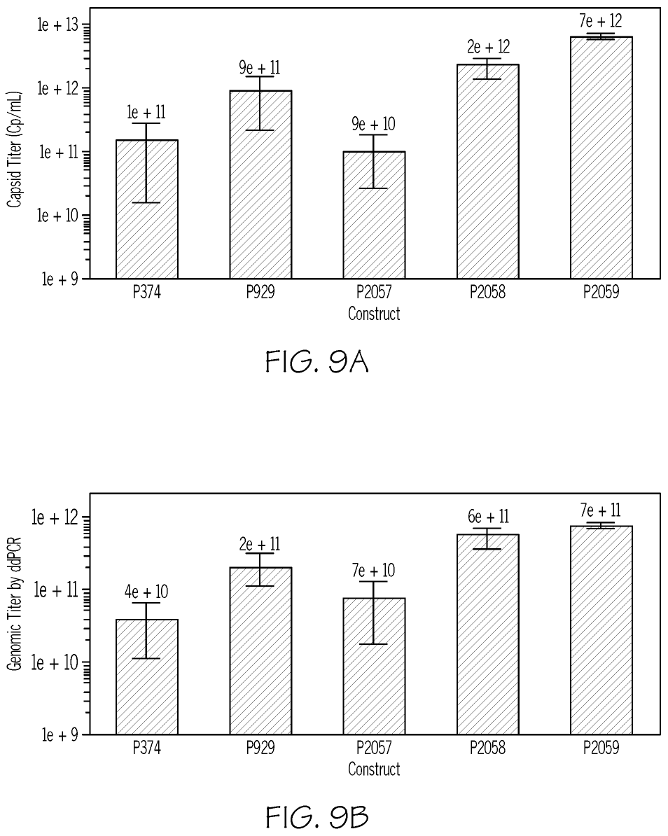

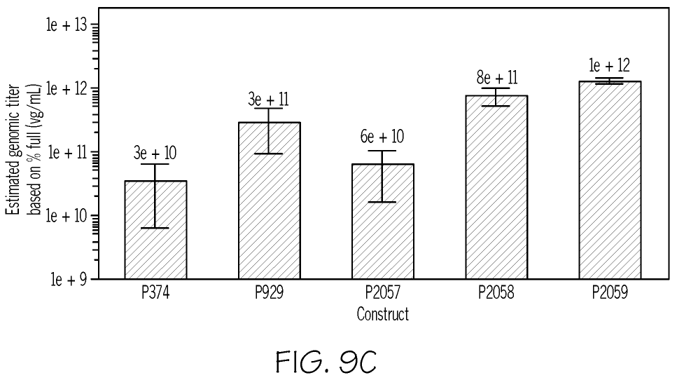

Each of the five plasmids was used to produce AAV1 vectors carrying an eGFP transgene. The vectors were produced in biological duplicates in the absence of doxycycline. A plasmid ratio of 2:1:2 with respect to—cap encoding plasmid:helper plasmid:eGFP plasmid was used for productions. FIGS. 9A to 9C depict production titer of AAV1 containing an enhanced green fluorescent protein (eGFP) transgene using plasmids P374, P929, P2057, P2058 or P2059. The same pHelper plasmid (p376) was used. Productions were conducted in 125 mL shake flasks using method described in Example 3.

FIG. 9A compares capsid titer measured by ELISA in clarified lysate samples for each of the five plasmids. P2059 achieved highest capsid titer (Cp/ml), followed by P2058 and P929. P2057 achieved slightly lower capsid titer compared to P374.

FIG. 9B compares genomic titer using droplet digital PCR (ddPCR) in clarified lysate samples for each of the five plasmids. The results are similar to FIG. 9A. All tetracycline regulated constructs (P929, P2057, P2058 and P2059), which can be regulated by tetracycline and derivatives like doxycycline, achieved higher genomic titer (vg/ml) than the P374 control. P2059 performed the best in terms of genomic titer, followed by P2058, P929 and P2057.

FIG. 9C compares estimated genomic titer based on percent full capsids for each of the five plasmids. The results are similar to FIG. 9B.

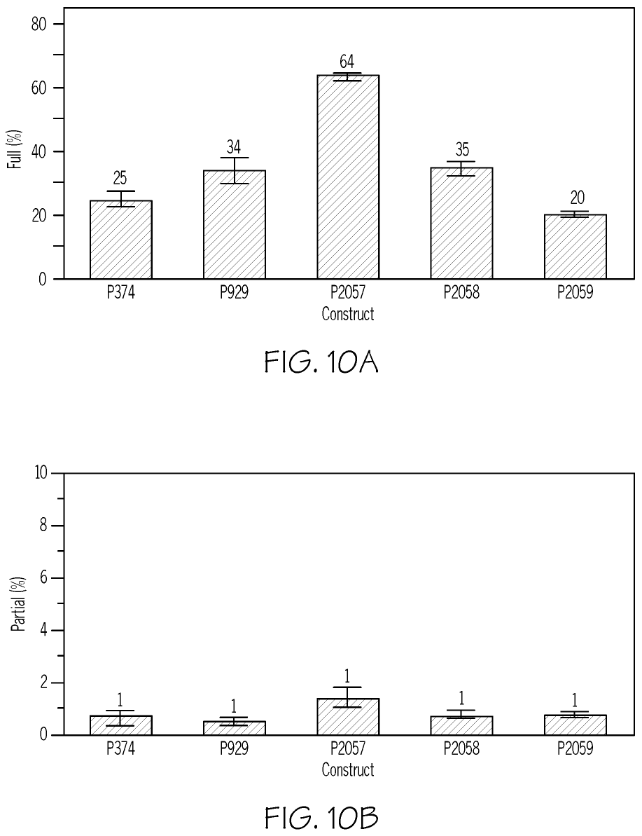

FIGS. 10A to 10B depict production of AAV1 containing an eGFP transgene using plasmids P374, P929, P2057, P2058 or P2059, and employ mass photometry to assess capsid fullness achieved by each of the five plasmids. Prior to the quantification of capsid fullness using mass photometry, the clarified lysate samples were purified.

FIG. 10A sets forth the percentage of full capsids. P2057 showed the highest percent full at 64%, followed by P2058 at 35%, and P929 at 34%. The percent full of vectors produced with P2059 was slightly lower than P374 control.

FIG. 10B sets forth the percentage of partially-filled capsids. P2057 showed the highest percentage. Vectors produced with all constructs showed minimal partially-filled capsids. The tabulated data is below in Table 7.

TABLE 7
Tabulated Data for a Comparison of
P374, P929, P2057, P2058 and P2059

| | | Clarified | % Full | % Partial | |
|---|---|---|---|---|---|
| | | Lysate | based on | based on | Estimated |
| | | Capsid | Mass | Mass | ddPCR titer |
| | Rep/Cap | Titer | Photom- | Photom- | (based on % |
| Sample | Plasmid | (cp/mL) | etry | etry | Full) |
| CER5707 | P374 | 5.30E+10 | 27.2 | 0.8 | 1.44E+10 |
| CER5708 | P374 | 2.40E+11 | 23.1 | 0.4 | 5.54E+10 |
| CER5715 | P929 | 1.30E+12 | 31.6 | 0.4 | 4.11E+11 |
| CER5716 | P929 | 4.10E+11 | 37 | 0.6 | 1.52E+11 |
| CER5723 | P2057 | 4.60E+10 | 63 | 1.2 | 2.90E+10 |
| CER5724 | P2057 | 1.40E+11 | 64.2 | 1.7 | 8.99E+10 |
| CER5731 | P2058 | 1.80E+12 | 36.3 | 0.9 | 6.53E+11 |
| CER5732 | P2058 | 2.90E+12 | 33.3 | 0.7 | 9.66E+11 |
| CER5739 | P2059 | 6.20E+12 | 20.3 | 0.8 | 1.26E+12 |
| CER5740 | P2059 | 7.10E+12 | 19.4 | 0.6 | 1.38E+12 |

The data in Example 7 is compelling. P2059 and 2058 performed the best in terms of overall capsid and genomic titer. However, in terms of percent full capsids, P2057 performed the best. Thus, where purification of full capsids is readily attained, P2059 and P2058 are a logical choice. However, where purification of full capsids is more difficult, P2057 can be the best choice regardless of its lower performance in terms of overall capsid and genomic titer.

### Other Inventions

While the present inventions have been described in connection with specific examples thereof, it will be understood that it is capable of further modifications and this application is intended to cover any variations, uses, or adaptations of the inventions following, in general, the principles of the inventions and including such departures from the inventions that come within known or customary practice within the art to which the inventions pertain and may be applied to the essential features hereinbefore set forth, and follow in the scope of the claims.

1. An adeno-associated virus (AAV) rep-cap plasmid comprising, in a 5′ to 3′ orientation:
(a) a polynucleotide encoding an AAV rep protein;
(b) a tetracycline-off (Tet-Off) system or a tetracycline-on (Tet-On) system;
(c) an AAV promoter; and
(d) a polynucleotide encoding an AAV capsid protein.

2. The rep-cap plasmid of claim 1, wherein the AAV capsid protein is an AAV1 capsid protein and the AAV promoter is an AAV P41 promoter or a TRE-tight promoter.

3-4. (canceled)

5. The rep-cap plasmid of claim 2, wherein the AAV rep protein is an AAV2 rep protein, wherein the polynucleotide encoding the AAV rep protein has the sequence of SEQ ID NO: 3.

6. The rep-cap plasmid of claim 2, wherein the polynucleotide encoding an AAV rep protein encodes Rep78 and Rep52, wherein Rep78 has the sequence of SEQ ID NO: 1, and wherein Rep52 has the sequence of SEQ ID NO: 2.

7-9. (canceled)

10. The rep-cap plasmid of claim 2, wherein the Tet-Off system comprises, in a 5′ to 3′ orientation:
(a) a first polynucleotide encoding a tetracycline trans-activator (tTA), wherein the tTA comprises a tetracycline-repressor-derived DNA binding domain and a transcriptional activation domain; and
(b) a second polynucleotide comprising 1 to 12 tet operator (tetO) sequences.

11. The rep-cap plasmid of claim 10, wherein the first polynucleotide encodes a polypeptide having an amino acid sequence of SEQ ID NO: 5 or SEQ ID NO: 20, and wherein the first polynucleotide has at least 80% sequence identity to SEQ ID NO: 6 or SEQ ID NO: 21.

12-13. (canceled)

14. The rep-cap plasmid of claim 10, wherein each tetO sequence has the sequence of SEQ ID NO: 4, wherein each tetO sequence is separated from an adjacent tetO sequence by 0-20 nucleotides.

15. (canceled)

16. The rep-cap plasmid of claim 10, wherein the second polynucleotide comprises eight tetO sequences, and wherein the second polynucleotide has at least 80% sequence identity to SEQ ID NO: 7.

17-27. (canceled)

28. The rep-cap plasmid of claim 2, wherein the p41 promoter has the sequence of SEQ ID NO: 8.

29. The rep-cap plasmid of claim 2, wherein the polynucleotide encoding an AAV1 capsid protein encodes a polypeptide having an amino acid sequence of SEQ ID NO: 9, SEQ ID NO: 10, or SEQ ID NO: 11.

30-31. (canceled)

32. The rep-cap plasmid of claim 2, wherein the Tet-Off system or Tet-On system further comprises a polyA signal sequence located between the first polynucleotide and the second polynucleotide.

33. The rep-cap plasmid of claim 2, comprising a polynucleotide sequence of SEQ ID NO: 12, SEQ ID NO: 13, or SEQ ID NO: 22.

34-35. (canceled)

36. A method of manufacturing a recombinant adeno-associated virus (rAAV) comprising the steps of:
(I) introducing into a mammalian cell:
(a) a rep-cap plasmid of claim 1;
(b) a helper plasmid comprising one or more helper genes selected from E4, E2a and VA; and
(c) a transgene plasmid comprising a transgene of interest flanked by inverse terminal repeats,
wherein the introducing step is under conditions that permit formation of the rAAV; and
(II) collecting the rAAV.

37. The method of claim 36, wherein the AAV promoter is an AAV P41 promoter or a TRE-tight promoter.

38. The method of claim 37, wherein the mammalian cell is a HEK-293 cell.

39. The method of claim 37, wherein the rep-cap plasmid comprises a polynucleotide sequence of SEQ ID NO: 12, SEQ ID NO: 13, or SEQ ID NO: 22.

40-41. (canceled)

42. The method of claim 37, wherein the transgene of interest encodes a protein that is normally expressed in the human ear, wherein the transgene of interest encodes solute carrier family 26, member 4 (Pendrin), Otoferlin (Otof), Stereocilin (STRC), Atonal BHLH Transcription Factor 1 (ATOH1), gap junction protein beta 2 (GJB2), or SRY-Box 2 (Sox2).

43-44. (canceled)

45. The method of claim 37, wherein the viral yield is at least 1×10¹¹ vg/ml as measured using ddPCR of clarified lysate.

46. The method of claim 37, wherein:
(a) the rep-cap plasmid comprises a Tet-On system; and
(b) the production of the recombinant AAV1 virus is achieved without inducing the Tet-On system.

47. A method of increasing the yield of a recombinant AAV1-capped adeno-associated virus by at least 2.5-fold over a recombinant AAV1-capped adeno-associated virus produced with pAAV-RC1, comprising the step of substituting a rep-cap plasmid of claim 2 for pAAV-RC1 in a manufacture of the adeno-associated virus.

48. (canceled)

49. The method of claim 47, wherein the rep-cap plasmid comprises a polynucleotide sequence of SEQ ID NO: 12, SEQ ID NO: 13, or SEQ ID NO: 22.

50-51. (canceled)

52. The rep-cap plasmid according to claim 2, wherein the rep-cap plasmid has an arrangement according to FIG. 1, FIG. 5, FIG. 6, or FIG. 7.

53-54. (canceled)

55. A cell comprising a rep-cap plasmid according to claim 2.

56. The cell according to claim 55, wherein the cell further comprises a transgene of interest that encodes a protein that is normally expressed in the human ear, wherein the transgene of interest encodes solute carrier family 26; member 4 (Pendrin); Otoferlin (Otof); Stereocilin (STRC); Atonal BHLH Transcription Factor 1 (ATOH1); gap junction protein beta 2 (GJB2); SRY-Box 2 (Sox2); GJB6; WFS1; COCH; EYA4; MYO7A; POU4F3; ACTG1; MY06; REST; NLRP3; COL11A1; TJP2; TBC1D24; SLC26A4; ELMOD3; EPSN; WHRN; IGF1; IGF1R; MYO15A; TMIE; TMC1; TMC2; TMPRSS3; OTOF; CDH23; GIPC3; USH1C; OTOG; TECTA; OTOA; PDCH15; CLDN14; WHRN; ESRRB; MYO3A; HGF, ILDR1, ADCY1; CIB2; MARVELD2; SOX2; SLC26A5; COL4A3; COL4A4; COL4A5; CLPP; PJVK; LRTOMT/COMT2; LOXHD1; TPRM; SYNE4; KCNJ10; PTPRQ; OTOGL; LHFPL5; A1PR2; CABP2; MET; GRXCR2; EPS8; CLIC5; EPS8L2; WBP2; ROR1; CLDN9; USHIG; PKHD1L1; DIAPH3; NDP; USH2A; CLRN1; SANS; HARS1; or TRIOBP.

57. (canceled)

58. The cell according to claim 55, wherein the cell is a mammalian cell.

59. The cell according to claim 58, wherein the mammalian cell is a HEK-293 cell.

60-72. (canceled)


SEQUENCE LISTING (22 sequences)

SEQ ID NO: 1 - protein, 621 aa, synthetic construct
  REGION 1..621: note = Synthetic: Rep78 protein
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERLNQNSNICFTHGVKDCLECFPVSESQPVSVVKKAYQKLCYIHHIMGKVPDACTACDLVNVDLDDCVSEQ

SEQ ID NO: 2 - protein, 397 aa, synthetic construct
  REGION 1..397: note = Synthetic: Rep52 protein
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERLNQNSNICFTHGVKDCLECFPVSESQPVSVVKKAYQKLCYIHHIMGKVPDACTACDLVNVDLDDCVSEQ

SEQ ID NO: 3 - DNA, 1863 bp, synthetic construct
  misc_feature 1..1863: note = Synthetic: polynucleotide encoding an AAV rep protein
  source 1..1863: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaa

SEQ ID NO: 4 - DNA, 19 bp, synthetic construct
  misc_feature 1..19: note = Synthetic: tetO sequence
  source 1..19: mol_type = other DNA
tccctatcagtgatagaga

SEQ ID NO: 5 - protein, 248 aa, synthetic construct
  REGION 1..248: note = Synthetic: tTA protein
MSRLDKSKVINGALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 6 - DNA, 747 bp, synthetic construct
  misc_feature 1..747: note = Synthetic: polynucleotide sequence encoding tTA protein of SEQ ID NO: 5
  source 1..747: mol_type = other DNA
atgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 7 - DNA, 278 bp, synthetic construct
  misc_feature 1..278: note = Synthetic: portion of the tetracycline-inducible expression system containing the eight tetO sequences
  source 1..278: mol_type = other DNA
tccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagaga

SEQ ID NO: 8 - DNA, 295 bp, synthetic construct
  misc_feature 1..295: note = Synthetic: p41 promoter
  source 1..295: mol_type = other DNA
tgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgct

SEQ ID NO: 9 - protein, 736 aa, unidentified
  REGION 1..736: note = AAV1 capsid protein VP1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - DNA, 2211 bp, synthetic construct
  misc_feature 1..2211: note = Synthetic: polynucleotide encoding an AAV1 capsid protein
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 11 - DNA, 2208 bp, synthetic construct
  misc_feature 1..2208: note = Synthetic: polynucleotide encoding an AAV1 capsid protein
  source 1..2208: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 12 - DNA, 9309 bp, synthetic construct
  misc_feature 1..9309: note = Synthetic: modified rep-cap plasmid
  source 1..9309: mol_type = other DNA
aaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaataattgcttgttaatcaataaaccgtttaattcgtttcagttgaactttggtctctgcgaagggcgaattcgttt

SEQ ID NO: 13 - DNA, 9306 bp, synthetic construct
  misc_feature 1..9306: note = Synthetic: modified rep-cap plasmid
  source 1..9306: mol_type = other DNA
aaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattgcttgttaatcaataaaccgtttaattcgtttcagttgaactttggtctctgcgaagggcgaattcgttt

SEQ ID NO: 14 - protein, 599 aa, unidentified
  REGION 1..599: note = AAV1 capsid protein VP2
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 533 aa, unidentified
  REGION 1..533: note = AAV1 capsid protein VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - DNA, 24 bp, synthetic construct
  misc_feature 1..24: note = Synthetic: forward primer
  source 1..24: mol_type = other DNA
ttgtgaaagattgactggtattct

SEQ ID NO: 17 - DNA, 18 bp, synthetic construct
  misc_feature 1..18: note = Synthetic: reverse primer
  source 1..18: mol_type = other DNA
aggcattaaagcagcgta

SEQ ID NO: 18 - DNA, 21 bp, synthetic construct
  misc_feature 1..21: note = Synthetic: probe
  source 1..21: mol_type = other DNA
taactatgttgctccttttac

SEQ ID NO: 19 - DNA, 72 bp, synthetic construct
  misc_feature 1..72: note = Synthetic: sequence in the SLC26A4 gene which encodes the pendrin protein
  source 1..72: mol_type = other DNA
aagggatggcatatgccctactagctgcagttcctgtcggatatggtctctactctgcttttttccctatcc

SEQ ID NO: 20 - protein, 248 aa, synthetic construct
  REGION 1..248: note = Synthetic: rtTA protein
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 21 - DNA, 747 bp, synthetic construct
  misc_feature 1..747: note = Synthetic: polynucleotide sequence encoding rtTA protein of SEQ ID NO: 20
  source 1..747: mol_type = other DNA
atgagtcggctggataaatctaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 22 - DNA, 9309 bp, synthetic construct
  misc_feature 1..9309: note = Synthetic: modified rep-cap plasmid
  source 1..9309: mol_type = other DNA
aaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaataattgcttgttaatcaataaaccgtttaattcgtttcagttgaactttggtctctgcgaagggcgaattcgttt